(19) United States
(12) Patent Application Publication
(10) Pub. No.: US 2024/0191292 A1
(43) Pub. Date: Jun. 13, 2024

(54) FLOW CELLS WITH PASSIVATION COMPONENTS

(71) Applicant: ILLUMINA, INC., San Diego, CA (US)

(72) Inventors: Johan Sebastian Basuki, Singapore (SG); Hassan Bohra, Singapore (SG); Hui Kheng Karen Goh, Singapore (SG); Yin Nah Teo, Singapore (SG); Wei Wei, Singapore (SG)

(21) Appl. No.: 18/523,636

(22) Filed: Nov. 29, 2023

Related U.S. Application Data
(60) Provisional application No. 63/385,586, filed on Nov. 30, 2022.

(51) Int. Cl. C12Q 1/6874; B01L 3/00

(52) U.S. Cl. CPC C12Q 1/6874; B01L 3/502761; B01L 2200/0647; B01L 2200/16; B01L 2300/0861

(57) ABSTRACT

An example flow cell includes a substrate including depressions separated by interstitial regions; a polymeric hydrogel positioned within each of the depressions; a primer set attached to the polymeric hydrogel; and one of: a passivation component attached to the interstitial regions, or a passivation component attached to the polymeric hydrogel, or respective passivation components attached to each of the interstitial regions and the polymeric hydrogel.

### CROSS-REFERENCE TO RELATED APPLICATIONS

This application claims the benefit of U.S. Provisional Application Ser. No. 63/385,586, filed Nov. 30, 2022, the contents of which is incorporated by reference herein in its entirety.

### REFERENCE TO SEQUENCE LISTING

The Sequence Listing submitted herewith is hereby incorporated by reference in its entirety. The name of the file is ILI250B_IP-2394-US_Sequence_Listing.xml, the size of the file is 15,413 bytes, and the date of creation of the file is Nov. 15, 2023.

### BACKGROUND

Some biological and/or chemical vessels, such as assay plates and flow cells, include designated reaction areas, where surface chemistry that enables a desired interaction or reaction is localized. When a reactive species is introduced into the vessel, the reactive species interacts or reacts with the surface chemistry to create a detectable signal (e.g., an electrical signal or an optical signal). Sometimes, however, the reactive species becomes bound outside the reaction area and/or becomes bound within the reaction area but does not interact or react in the intended manner with the surface chemistry. These non-specifically bound reactive species may generate signals that are not actually indicative of the reactive species interacting or reacting with the surface chemistry, and thus may result in surface fouling.

### SUMMARY

A variety of flow cells and methods are disclosed herein that reduce or eliminate surface fouling.

One example flow cell includes depressions separated by interstitial regions, and a polymeric hydrogel and primer set positioned within the depressions. In this example flow cell, a passivation component is attached to the interstitial regions, the polymeric hydrogel, or both the interstitial regions and the polymeric hydrogel. The passivation component helps to reduce or eliminate non-specific binding of labeled nucleotides introduced for sequencing template strands attached to the primers in the depressions.

In one specific flow cell where the passivation component is attached to the polymeric hydrogel, the passivation component may be reversible in that it can transition between a collapsed state where it does not interfere with binding and an expanded state that does interfere with binding (i.e., an anti-fouling state). The anti-fouling state may be activated during wash processes in order to reduce or eliminate non-specific binding of labeled nucleotides during non-incorporation processes.

One example method utilizes a wash solution including a cyclodextrin additive. This wash solution washes away non-incorporated labeled nucleotides from the flow cell surface.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of a flow cell;

FIG. 1B is a cross-sectional and semi-schematic view of an example architecture of the flow cell of FIG. 1A;

FIG. 1C is a cross-sectional and semi-schematic view of another example architecture of the flow cell of FIG. 1A;

FIG. 1D is a cross-sectional and semi-schematic view of still another example architecture of the flow cell of FIG. 1A;

FIG. 2A is a cross-sectional and semi-schematic view of a depression including a reversible passivation component having a lower critical solution temperature (LCST) polymer or a pH responsive monomer co-polymerized with an anti-fouling monomer, both during and after labeled nucleotide incorporation;

FIG. 2B is a cross-sectional and semi-schematic view of a depression including a reversible passivation component having an upper critical solution temperature (UCST) polymer, both during and after labeled nucleotide incorporation;

FIG. 3 is a bar graph depicting the mean fluorescence intensity (arbitrary units, Y axis) for a non-passivated control flow cell (no PEG) and three example flow cells whose polymeric hydrogel was passivated with different molecular weight polyethylene glycol moieties after passivation, after incubation, and after washing;

FIG. 4 is a bar graph depicting the mean fluorescence intensity (arbitrary units, Y axis) for four non-passivated flow cells (no PEG) and four passivated flow cells (PEG) after passivation, after incubation, and after washing, where the flow cells were exposed to different washing solutions; and

FIG. 5 is a bar graph depicting the mean fluorescence intensity (arbitrary units, Y axis) for four passivated flow cells (PEG) after incubation and after washing, where the flow cells were exposed to different washing solutions.

### DETAILED DESCRIPTION

Surface fouling can occur in a biological and/or chemical vessel when a reactive species, such as a dye-labelled nucleotide, a protein, or another biomolecule, non-specifically binds outside the vessel's reaction area and/or within the vessel's reaction area but in a manner that does not interact or react with the intended surface chemistry. The surface is considered fouled because the non-specifically bound reactive species can generate signals that are not actually indicative of the reactive species interacting or reacting with the surface chemistry, and thus are considered noise. As such, these non-specifically bound reactive species can decrease the signal-to-noise ratio.

A variety of flow cells and methods are disclosed herein that reduce or eliminate surface fouling.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylate” is a salt, ester, or conjugate base of acrylic acid:

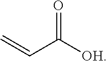

An “acrylamide” is a functional group with the structure

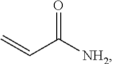

where each H may alternatively be an alkyl, an alkylamino, an alkylamido, an alkylthio, an aryl, a glycol, and optionally substituted variants thereof.

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-C6 alkyl” indicates that there are one to six carbon atoms in the alkyl chain, i.e., the alkyl chain is selected from the group consisting of methyl, ethyl, propyl, iso-propyl, n-butyl, isobutyl, sec-butyl, t-butyl, pentyl, and hexyl.

As used herein, “alkylamino” refers to an alkyl group in which one or more of the hydrogen atoms are replaced by an amino group, where the amino group refers to an —NRₐR_{b} group, where Rₐ and R_{b} are each independently selected from a C1-C6 alkyl, C2-C6 alkenyl, C2-C6 alkynyl, C3-C7 carbocycle, C6-C10 aryl, a 5-10 membered heteroaryl, and a 5-10 membered heterocycle.

As used herein, “alkylamido” refers to an alkyl group in which one or more of the hydrogen atoms are replaced by a C-amido group or an N-amido group. A “C-amido” group refers to a “—C(═O)N(RₐR_{b})” group in which Rₐ and R_{b} can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. An “N-amido” group refers to a “RC(═O)N(Rₐ)—” group in which R and Rₐ can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. Any alkylamido may be substituted or unsubstituted.

As used herein, “alkylthio” refers to RS—, in which R is an alkyl. The alkylthio can be substituted or unsubstituted.

As used herein, “alkene” or “alkylene” or “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

An “alkoxy” group refers to the formula —OR, wherein R is an alkyl, an alkenyl, an alkynyl, a cycloalkyl, a cycloalkenyl or a cycloalkynyl as defined herein. Some example alkoxy groups include methoxy, ethoxy, n-propoxy, 1-methylethoxy (isopropoxy), n-butoxy, iso-butoxy, sec-butoxy, and tert-butoxy. Any alkoxy may be substituted or unsubstituted.

As used herein, “alkyne” or “alkynyl” refers to a straight or branched hydrocarbon chain containing one or more triple bonds. The alkynyl group may have 2 to 20 carbon atoms.

An “allyl” refers to the unsaturated hydrocarbon radical —CH═CHCH₂.

As used herein, an “amphoteric polymer” is a co-polymer that contains both cationic and anionic monomers and that can function as both an acid and a base.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl, and naphthylalkyl.

The term “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl. Any aryl may be a heteroaryl, with at least one heteroatom, that is, an element other than carbon (e.g., nitrogen, oxygen, sulfur, etc.), in ring backbone.

As used herein, the term “attached” refers to the state of two things being joined, fastened, adhered, connected or bound to each other, either directly or indirectly. For example, a nucleic acid can be attached to a functionalized polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl. Any of the carbocycles may be heterocycles, with at least one heteroatom in ring backbone.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Still another example is dibenzocyclooctyne (DBCO).

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate or a patterned material having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

An “end group” of any example of a passivation component is a terminal functional group or polymer that exhibits an anti-fouling property (i.e., reduces or prevents non-specific adsorption of nucleotides or other biomolecules).

As used herein, the term “flow cell” is intended to mean a vessel having a flow channel that is in fluid communication with surface(s) containing surface chemistry. In some instances, the flow cell surface chemistry is removable and/or regenerable, thus rendering the surface reusable. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components or defined in an open component, which can selectively receive a liquid sample. In some examples, the flow channel may be defined between a patterned or non-patterned structure and a lid. In other examples, the flow channel may be defined between two patterned or non-patterned structures that are bonded together. In still further examples, the flow channel is defined in a structure and is open to the external environment.

As used herein, “heteroalicyclic” or “heteroalicycle” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heteroalicyclic ring system may optionally contain one or more unsaturated bonds situated in such a way, however, that a fully delocalized pi-electron system does not occur throughout all the rings. The heteroatoms are independently selected from oxygen, sulfur, and nitrogen. A heteroalicyclic ring system may further contain one or more carbonyl or thiocarbonyl functionalities, so as to make the definition include oxo-systems and thio-systems such as lactams, lactones, cyclic imides, cyclic thioimides, and cyclic carbamates. The rings may be joined together in a fused fashion. Additionally, any nitrogens in a heteroalicyclic may be quaternized. Heteroalicycle or heteroalicyclic groups may be unsubstituted or substituted. Examples of such “heteroalicyclic” or “heteroalicycle” groups include 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

A “(heteroalicyclic)alkyl” refers to a heterocyclic or a heteroalicyclic group connected, as a substituent, via a lower alkylene group. The lower alkylene and heterocycle or a heterocycle of a (heteroalicyclic)alkyl may be substituted or unsubstituted. Examples include tetrahydro-2H-pyran-4-yl)methyl, (piperidin-4-yl) ethyl, (piperidin-4-yl)propyl, (tetrahydro-2H-thiopyran-4-yl)methyl, and (1,3-thiazinan-4-yl)methyl.

The term “hydrazine” or “hydrazinyl” as used herein refers to a —NHNH₂ group.

As used herein, the term “hydrazone” or “hydrazonyl” as used herein refers to a

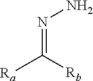

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a substrate, patterned resin, or other support that separates depressions. For example, an interstitial region can separate one depression of an array from another depression of the array. The two depressions that are separated from each other can be discrete, i.e., lacking physical contact with each other. In many examples, the interstitial region is continuous whereas the depressions are discrete, for example, as is the case for a plurality of depressions defined in an otherwise continuous surface. In other examples, the interstitial regions and the features (e.g., depressions) are discrete, for example, as is the case for a plurality of trenches separated by respective interstitial regions. The separation provided by an interstitial region can be partial or full separation. Interstitial regions may have a surface material that differs from the surface material of the depressions. For example, the depression can include the polymeric hydrogel, while the interstitial regions are free of the polymeric hydrogel.

The term “(meth)” in conjunction with a functional group means that one of the hydrogen atoms of the functional group may be replaced with a methyl group. For example, (meth)acrylate means either acrylate or methacrylate.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acids (PNA).

A “passivation component” is a molecule including an end group that exhibits an anti-fouling property.

“Perfluorinated” refers to an organofluorine compound containing only carbon-fluorines and C—C bonds, and in some instances heteroatoms.

“Positively chargeable” refers to a molecule that is capable of carrying a positive charge.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of a primer set may be modified to allow a coupling reaction with a functional group of one of the orthogonal polymers. The primer length can be any number of bases long and can include a variety of non-natural nucleotides. In an example, the sequencing primer is a short strand, ranging from 10 to 60 bases, or from 20 to 40 bases.

The “propargyl group” is an alkyl functional group of 2-propynyl with the structure HC≡C—CH₂—.

The terms “silane small molecule,” “siloxane small molecule,” and alkoxy silane small molecule” refer to molecules having a weight average molecular weight ranging from about 100 Da to about 1,000 Da and respectively including a siloxane (Si—O—Si linkage), a silane (SiH₄) or an alkoxy silane (Si(OR)₄, where R is an alkyl or an aryl).

The term “substrate” refers to a structure upon which various components of the flow cell (e.g., polymeric hydrogel, primers, etc.) may be added. The substrate may be a wafer, a panel, a rectangular sheet, a die, or any other suitable configuration. The substrate is generally rigid and is insoluble in an aqueous liquid. The substrate may be a single layer structure, or a multi-layered structure (e.g., including a support and a patterned material on the support). Examples of suitable substrates will be described further herein.

The term “sulfonic acid” refers to R—S(═O)₂—OH.

As used herein, the terms “tetrazine” and “tetrazinyl” refer to six-membered heteroaryl group comprising four nitrogen atoms. Tetrazine can be optionally substituted.

“Tetrazole,” as used herein, refers to a five-membered heterocyclic group including four nitrogen atoms. Tetrazole can be optionally substituted.

“Zwitterionic” refers to a molecule having a net formal charge of zero, but negative and positive formal charges on individual atoms within its structure.

### Flow Cells

Examples of the flow cell disclosed herein generally include a substrate including depressions separated by interstitial regions; a polymeric hydrogel positioned within each of the depressions; a primer set attached to the polymeric hydrogel; and one of: a passivation component attached to the interstitial regions, a passivation component attached to the polymeric hydrogel, or respective passivation components attached to each of the interstitial regions and the polymeric hydrogel. In some examples, the passivation component is reversible in that it is switchable between a collapsed state where it does not interfere with binding and an expanded state where it does interfere with binding (i.e., an anti-fouling state).

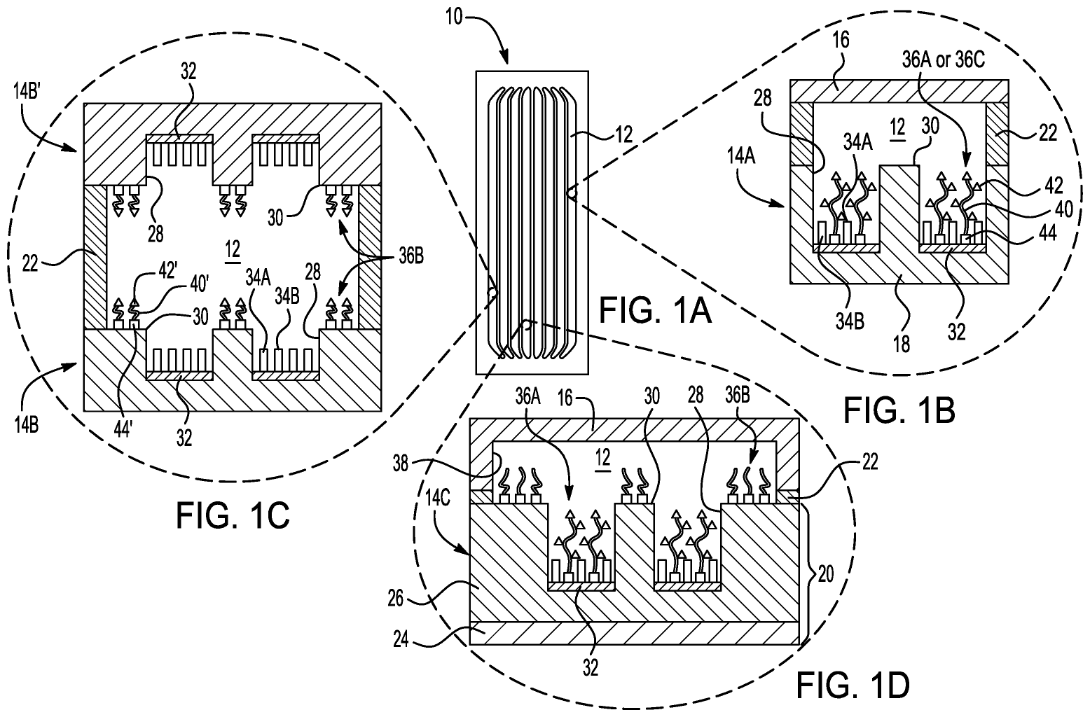

A top view of a flow cell 10 is shown in FIG. 1A, and examples of the architecture within a flow channel 12 of the flow cell 10 are shown in FIG. 1B, FIG. 1C, and FIG. 1D. These example architectures may include one patterned structure 14A bonded to a lid 16 (see FIG. 1B and FIG. 1D) or two patterned structures 14B, 14B′ bonded together (see FIG. 1C). It is to be understood that the patterned structure 14A in FIG. 1B or the patterned structure 14C in FIG. 1D may alternatively be bonded to another patterned structure, and the patterned structure 14B in FIG. 1C may alternatively be bonded to the lid 16. In still other examples, the flow cell architecture may include a single patterned structure 14A, 14B, or 14C without an additional structure or lid bound thereto. In this example, the flow channel 12 and the surface chemistry of the single patterned structure 14A, 14B, 14C are open to the external environment.

In some examples, the flow channel 12 is enclosed and is defined between the one patterned structure 14A, 14B, or 14C and the lid 16 or between two of the patterned structures, e.g., 14B and 14B′. In other examples, the flow channel 12 is open and is defined in an area of the single patterned structure 14A, 14B, or 14C where the depressions 28 are defined.

The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). When multiple flow channels 12 are included, each flow channel 12 may be isolated from each other flow channel 12 so that fluid introduced into a flow channel 12 does not flow into adjacent flow channel(s) 12.

Each flow channel 12 is in fluid communication with an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) which controls fluid introduction and expulsion. Some examples of the fluids introduced into the flow channel 12 may introduce reaction components (e.g., DNA sample, polymerases, sequencing primers, labelled nucleotides, etc.), washing agents, deblocking agents, etc. In the open form of the flow cell 10, the fluid introduction and extraction may be manual and thus the flow channel 12 may not include a designated inlet and outlet.

The flow channel 12 may have any desirable shape. In an example, the flow channel 12 has a substantially rectangular configuration with curved ends (as shown in FIG. 1A). The length of the flow channel 12 depends, in part, upon the size of the substrate (e.g., 18 or 20, see FIG. 1B through FIG. 1D) in which the patterned structure 14A, 14B, 14C is formed. The width of the flow channel 12 depends, in part, upon the size of the substrate 18, 20 in which the patterned structure 14A, 14B, 14C is formed, the desired number of flow channels 12, the desired space between adjacent channels 12, and the desired space at a perimeter of the patterned structure 14A, 14B, 14C. The spaces between channels 12 and at the perimeter may be sufficient for attachment to the lid 16 or the patterned structure 14B′.

The depth of the flow channel 12 can be as small as a monolayer thick when microcontact, aerosol, or inkjet printing is used to deposit a separate material (e.g., a spacer layer 22) that defines at least a portion of the side walls of the flow channel 12. In other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

The spacer layer 22 used to attach the lid 16 and the patterned structure 14A, 14B, 14C or two patterned structures, e.g., 14B and 14B′, may be any material that will seal portions of the lid 16 and the patterned structure 14A, 14B, 14C together or portions of the two patterned structures, e.g. 14B and 14B′, together. As examples, the spacer layer 22 may be an adhesive, a radiation-absorbing material that aids in bonding, or the like. In some examples, the spacer layer 22 is the radiation-absorbing material, e.g., KAPTON® black (available from DuPont de Nemours).

The lid 16 may be any material that is transparent to the excitation light that is directed toward the flow cell 10. In optical detection systems, the lid 16 may also be transparent to the emissions generated from reaction(s) taking place in the flow cell 10. As examples, the lid 16 may include glass (e.g., borosilicate, fused silica, etc.) or a transparent polymer. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P. In some instances, the lid 16 is shaped to form the top of the flow cell 10 (e.g., as shown in FIG. 1B), and in other instances, the lid 16 is shaped to form both the top of the flow cell 10 as well as sidewalls 38 of the flow channel 12 (e.g., as shown in FIG. 1D).

The lid 16 and the patterned structure 14A, 14B, 14C or two patterned structures, e.g., 14B and 14B′ are sealed together at designated bonding regions, which are located at the perimeter of the substrate 18, 20. Additionally, in multi-channel flow cells 10, a bonding region is also located at the perimeter of each flow channel 12. Thus, in these examples, the flow channel 12 is defined by the patterned structure 14A, 14B, 14C, the spacer layer 22, and either the lid 16 or the other patterned structure, e.g., 14B′.

Each example of the patterned structure 14A, 14B, 14C includes a substrate 18 or 20. The substrate 18 is a single layer base support (as shown in FIG. 1B and FIG. 1C). The substrate 20 is a multi-layered structure (as shown in FIG. 1D).

Examples of suitable single layer base support (i.e., substrate 18) materials include epoxy siloxane, glass, modified or functionalized glass, polymeric materials (including acrylics, polystyrene and co-polymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, nylon (polyamides), etc.), ceramics/ceramic oxides, silica (i.e., silicon dioxide (SiO₂)), fused silica, or silica-based materials, aluminum silicate, silicon and modified silicon (e.g., boron doped p+ silicon), silicon nitride (Si₃N₄), tantalum pentoxide (Ta₂O₅) or other tantalum oxide(s) (TaOₓ), hafnium oxide (HfO₂), carbon, metals, or the like. In some examples, the resins set forth hereinbelow may also be used as the single layer substrate 18.

Examples of the multi-layered structure (i.e., substrate 20) include a base support 24 and at least one other layer 26 thereon. Any example of the single layer base support (i.e., substrate 18) may be used as the base support 24. The other layer 26 may be any material that can be etched or imprinted to form depressions 28. Examples of the layer 26 include inorganic oxides, such as tantalum oxide (e.g., Ta₂O₅), aluminum oxide (e.g., Al₂O₃), silicon oxide (e.g., SiO₂), or hafnium oxide (e.g., HfO₂), or polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

The substrate 18 or the layer 20 may include, or be functionalized to include, surface groups that are to attach to the passivation component 36B. In particular, these surface groups are exposed at the interstitial regions 30. Examples of the surface groups may include hydroxyl groups, epoxy groups, and norbornene groups. In one example, a glass substrate 18 may be silanized with norbornene silane to introduce norbornene surface groups that can attach to some examples of the passivation component 36B. These norbornene surface groups are alkene containing groups that can react with anchors, such as DBCO, bicyclo[6.1.0]nonyne (BCN), norbornyl functional groups, or propargyl functional groups. In another example, an epoxy functionalized polyhedral oligomeric silsesquioxane based resin layer 26 of the multi-layer substrate 20 may inherently include hydroxyl and epoxy surface groups that can attach to some examples of the passivation component 36B.

In any of the examples set forth herein, the single layer base support/substrate 18 and the base support 24 may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). As one example, a die may have a width ranging from about 0.1 mm to about 10 mm. While example dimensions have been provided, it is to be understood that the base supports 18, 24 may have any suitable dimensions.

Each example of the patterned structure 14A, 14B, 14C also includes depressions 28 defined in the substrate 18 or 20. Each depression 28 is a three-dimensional structure that extends inward (downward) from an adjacent surface, i.e., an interstitial region 30. The depression 28 is thus a concave region with respect to the interstitial regions 30 that surround the depression 28. Each depression 28 is formed in the substrate 18 or the layer 26 via etching, photolithography, imprinting, etc. so that the interstitial regions 30 completely surround the depression 28 (e.g., when the depressions 28 are formed as individual wells) or surround opposed sides of the depression 28 (e.g., when the depressions 28 are formed as trenches).

The depressions 28 may be formed in an array across the substrate 18 or the layer 26. In some examples, all of the depressions 28 defined in the substrate 18 or the layer 26 are in fluid communication with a single flow channel 12 of the flow cell 10. In some of these examples, interstitial regions 30 at the perimeter of the substrate 18 or the layer 26 provide the bonding region where the lid 16 or the second patterned structure 14A, 14B, 14C is attached to form the enclosed flow cell 10 having the single flow channel 12. In some other of these examples, interstitial regions 30 at the perimeter of the substrate 18 or the layer 26 provide a boundary for the open flow channel 12. In other examples, the flow cell 10 includes several discrete flow channels 12 (e.g., two, four, eight, etc.), and a respective subset of the depressions 28 is in fluid communication with each of the flow channels 12. In some of these examples, interstitial regions 30 between adjacent flow channels 12 provide the bonding regions where the lid 16 or the second patterned structure 14A, 14B, 14C is attached to form an enclosed flow cell having multiple flow channels. In some other of these examples, interstitial regions 30 between adjacent flow channels 12 provide the boundary for each open flow channel 12.

Whether arranged to be in fluid communication with a single or multiple flow channels 12, many different patterns for the depressions 28 are envisaged, including regular, repeating, and non-regular patterns. In an example, the depressions 28 are disposed in a hexagonal grid for close packing and improved density. Other layouts may include, for example, rectangular layouts, triangular layouts, striped layouts, diagonal layouts, etc. In some examples, the layout or pattern can be an x-y format of depressions 28 that are in rows and columns. In still other examples, the layout or pattern can be a random arrangement of depressions 28.

The layout or pattern of the depressions 28 may be characterized with respect to the density of the depressions 28 (e.g., number of depressions 28) in a defined area. For example, the depressions 28 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of depressions 28 can be between one of the lower values and one of the upper values selected from the ranges above. As examples, a high density array may be characterized as having depressions 28 separated by less than about 100 nm, a medium density array may be characterized as having depressions 28 separated by about 400 nm to about 1 μm, and a low density array may be characterized as having depressions 28 separated by greater than about 1 μm. While example densities have been provided, it is to be understood that any suitable densities may be used. In some instances, it may be desirable for the spacing between depressions 28 to be even greater than the examples listed herein.

The layout or pattern of the depressions 28 may also or alternatively be characterized in terms of the average pitch, or the spacing from the center of one depression 28 to the center of an adjacent depression 28 (center-to-center spacing) or from the left edge of one depression 28 to the right edge of an adjacent depression 28 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of depressions 28 can be between one of the lower values and one of the upper values selected from the ranges above. In an example, the depressions 28 have a pitch (center-to-center spacing) of about 1.5 μm. While example average pitch values have been provided, it is to be understood that other average pitch values may be used.

While any suitable three-dimensional geometry may be used for the depressions 28, a geometry with an at least substantially flat bottom surface may be desirable so that a layer of the polymeric hydrogel 32 may be formed thereon. Example depression geometries include a sphere, a cylinder, a cube, polygonal prisms (e.g., rectangular prisms, hexagonal prisms, etc.), or the like.

Depressions 28 can have any of a variety of shapes at their opening including, as examples, round, elliptical, square, polygonal, star shaped (with any number of vertices), etc. The cross-section of a depression 28 taken orthogonally with the interstitial region 30 can be curved, square, polygonal, hyperbolic, conical, angular, etc.

The size of each depression 28 may be characterized by its volume, opening area, depth, and/or diameter or length and width.

Each depression 28 can have any volume that is capable of receiving the polymeric hydrogel 32. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 28 is large enough to house at least the polymeric hydrogel 32 and primers 34A, 34B. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 34 can be greater than, less than or between the values specified above.

In some instances, the diameter or each of the length and the width of each depression 28 can be at least about 50 nm, at least about 0.1 μm, at least about 0.5 μm, at least about 1 μm, at least about 10 μm, at least about 100 μm, or more. Alternatively or additionally, the diameter or each of the length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or each of the length and width is about 0.4 μm. The diameter or each of the length and width of each depression 28 can be greater than, less than or between the values specified above.

Each of the patterned structures 14A, 14B, 14C includes a polymeric hydrogel 32 in the depressions 28. The polymeric hydrogel 32 may be any gel material that can swell when liquid is taken up and can contract when liquid is removed, e.g., by drying. In an example, the polymeric hydrogel 32 includes an acrylamide co-polymer. Some examples of the acrylamide co-polymer are represented by the following structure (I):

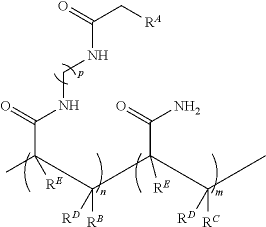

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxyl, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

It is to be understood that some of the R^{A} groups may attach the polymeric hydrogel 32 to the single layer substrate 18 or the layer 26. Some other of the R^{A} groups are the surface groups that can attach to the primers 34A, 34B, and in some examples, to the passivation component 36A.

One specific example of the acrylamide co-polymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of the acrylamide co-polymer may range from about 5 kDa to about 1500 kDa or from about 10 kDa to about 1000 kDa, or may be, in a specific example, about 312 kDa.

In some examples, the acrylamide co-polymer is a linear polymer. In some other examples, the acrylamide co-polymer is a lightly cross-linked polymer.

In other examples, the gel material may be a variation of structure (I). In one example, the acrylamide unit may be replaced with N,N-dimethylacrylamide

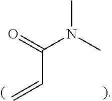

In this example, the acrylamide unit in structure (I) may be replaced with,

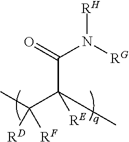

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

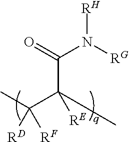

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 22, the recurring “n” feature in structure (I) may be replaced with a monomer including a heterocyclic azido group having structure (II):

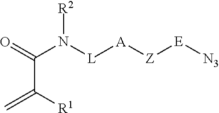

wherein R₁ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the gel material may include a recurring unit of each of structure (III) and (IV):

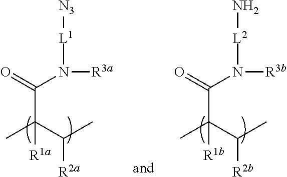

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide co-polymer is formed using nitroxide mediated polymerization, and thus at least some of the co-polymer chains have an alkoxyamine end group. In the co-polymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the co-polymer chain. In some examples, the alkoxyamine may also be introduced into some of the recurring acrylamide monomers, e.g., at position R^{A} in structure (I). As such, in one example, structure (I) includes an alkoxyamine end group; and in another example, structure (I) includes an alkoxyamine end group and alkoxyamine groups in at least some of the side chains.

It is to be understood that other molecules may be used to form the polymeric hydrogel 32, as long as they are capable of attaching to the single layer substrate 18 or the layer 26, the primers 34A, 34B, and in some examples, the passivation component 36A. Some examples of suitable materials for the polymeric hydrogel 32 include functionalized silanes, such as norbornene silane, azido silane, alkyne functionalized silane, amine functionalized silane, maleimide silane, or any other silane having functional groups that can achieve the desired attachments. Still other examples of suitable materials for the polymeric hydrogel 32 include those having a colloidal structure, such as agarose; or a polymer mesh structure, such as gelatin; or a cross-linked polymer structure, such as polyacrylamide polymers and co-polymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable materials for the polymeric hydrogel 32 include mixed co-polymers of acrylamides and acrylates. A variety of polymer architectures containing acrylic monomers (e.g., acrylamides, acrylates etc.) may be utilized in the examples disclosed herein, such as branched polymers, including dendrimers (e.g., multi-arm or star polymers). For example, the monomers (e.g., acrylamide, acrylamide containing the catalyst, etc.) may be incorporated, either randomly or in block, into the branches (arms) of a dendrimer.

The gel material for the polymeric hydrogel 32 may be formed using any suitable co-polymerization process, such as nitroxide mediated polymerization (NMP), reversible addition-fragmentation chain-transfer (RAFT) polymerization, etc.

Each example of the flow cell architecture includes the primers 34A, 34B attached to the polymeric hydrogel 32. The primers 34A, 34B are two different primers of a primer set that are used in sequential paired end sequencing. In sequential paired end sequencing, the primer set is used to amplify a nucleic acid (library) template molecule that has seeded to one of the two primers 34A, 34B or to a capture primer (not shown) attached in each of the depressions 28. In an example, forward strands are generated, sequenced and removed, and then reverse strands are generated, sequenced, and removed.

As examples, the primer set may include P5 and P7 primers, P15 and P7 primers, or any combination of the PA primers, the PB primers, the PC primers, and the PD primers set forth herein. As example combinations, the primer set may include any two PA, PB, PC, and PD primers, or any combination of one PA primer and one PB, PC, or PD primer, or any combination of one PB primer and one PC or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ ™, NEXTSEQDX™, NOVASEQ™, ISEQ™, GENOME ANALYZER™, and other instrument platforms.

The P5 primer may be:

P5 #1: 5′→3′
(SEQ. ID. NO. 1)
AATGATACGGCGACCACCGAGAUCTACAC,
or
P5 #2: 5′→3′
(SEQ. ID. NO. 2)
AATGATACGGCGACCACCGAGAnCTACAC

where “n” is alkene-thymidine (i.e., alkene-dT) in the sequence.

The P7 primer may be any of the following:

P7 #1: 5′→3′
(SEQ. ID. NO. 3)
CAAGCAGAAGACGGCATACGAnAT
P7 #2: 5′→3′
(SEQ. ID. NO. 4)
CAAGCAGAAGACGGCATACnAGAT
P7 #3: 5′→3′
(SEQ. ID. NO. 5)
CAAGCAGAAGACGGCATACnAnAT

where “n” is 8-oxoguanine in each of these sequences.

The P15 primer is:

P15: 5′→3′
(SEQ. ID. NO. 6)
AATGATACGGCGACCACCGAGAnCTACAC

where “n” is allyl T (a thymine nucleotide analog having an allyl functionality).

The other primers (PA-PD) mentioned above include:

PA 5′→3′
(SEQ. ID. NO. 7)
GCTGGCACGTCCGAACGCTTCGTTAATCCGTTGAG
PB 5′→3′
(SEQ. ID. NO. 8)
CGTCGTCTGCCATGGCGCTTCGGTGGATATGAACT
PC 5′→3′
(SEQ. ID. NO. 9)
ACGGCCGCTAATATCAACGCGTCGAATCCGCAACT
PD 5′→3′
(SEQ. ID. NO. 10)
GCCGCGTTACGTTAGCCGGACTATTCGATGCAGC

While not shown in the example sequences for PA-PD, it is to be understood that any of these primers may include a cleavage site, such as uracil, 8-oxoguanine, allyl-T, etc. at any point in the strand, as long as the cleavage sites of the primers 34A, 34B are orthogonal (i.e., the cleaving chemistry of the primer 34A is different than the cleaving chemistry for the primer 34B, and thus the two primers 34A, 34B are susceptible to different cleaving agents).

Each of the primers 34A, 34B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The immobilization of the primers 34A, 34B may be by single point covalent attachment at the 5′ end of the primers 34A, 34B. The 5′ terminal end of the primers 34A, 34B will vary depending upon the chemistry at the surface of the polymeric hydrogel 32. As two examples, the 5′ end functional groups of the primers 34A, 34B may be a terminal alkyne (e.g., hexynyl) or an internal alkyne, where the alkyne is part of a cyclic compound (e.g., bicyclo[6.1.0]nonyne (BCN)). The terminal alkynes can attach to azide groups at the surface of the polymeric hydrogel 32. In another example, the primers 34A, 34B may include an alkene at the 5′ terminus, which can react with reactive thiol groups at the surface of the polymeric hydrogel 32. In still other specific examples, succinimidyl (NHS) ester terminated primers may be reacted with amine groups at the surface of the polymeric hydrogel 32, aldehyde terminated primers may be reacted with hydrazine groups at the surface of the polymeric hydrogel 32, azide terminated primers may be reacted with an alkyne or DBCO (dibenzocyclooctyne) at the surface of the polymeric hydrogel 32, or amino terminated primers may be reacted with activated carboxylate groups at the surface of the polymeric hydrogel 32.

While not shown in FIG. 1B, FIG. 1C, or FIG. 1D, it is to be understood that each depression 28 may also include a PX primer for capturing a library template seeding molecule. The density of the PX motifs should be relatively low in order to minimize polyclonality.

The PX capture primers may be:

PX 5′→3′
(SEQ. ID. NO. 11)
AGGAGGAGGAGGAGGAGGAGGAGG
cPX (PX') 5′→3′
(SEQ. ID. NO. 12)
CCTCCTCCTCCTCCTCCTCCTCCT

In the example shown in FIG. 1B, the passivation component 36A is attached to the polymeric hydrogel 32 in at least some of the depressions 28.

The passivation component 36A includes a linker 40 and an end group 42, which functions as an anti-fouling agent, attached to one end of the linker 40. Multiple end groups 42 may also be attached to side chains or branches of the linker 40, and thus these particular groups 42 are not positioned at the opposed terminal ends of the passivation component 36A. The other end of the linker 40 is capable of attaching to the polymeric hydrogel 32, e.g., through an anchor 44. The anchor 44 may be the end group of the linker 40 or may be a separate molecule that is attached to the linker 40.

In the example where the passivation component 36A is exclusively attached to the polymeric hydrogel 32, the passivation component 36A includes i) the linker 40, which is selected from the group consisting of alkylene, poly(ethylene glycol), poly(meth)acrylate, polyacrylamide, pentaerythritol, and combinations thereof, and the end group 42, which is selected from the group consisting of a poly(ethylene glycol) end group when the linker is poly(meth)acrylate, polyacrylamide, pentaerythritol, and combinations thereof, a zwitterionic end group, a hydroxyl end group, a carboxylic acid end group, a sulfonic acid end group, a positively chargeable end group, an alkoxy end group, an anionic polymer end group, and an amphoteric polymer end group, or ii) a dibenzocyclooctyne-functionalized passivation agent.

Examples of the alkylene linker include ethylene, propylene, and butylene. The poly(ethylene glycol) linker has a weight average molecular weight up to 10,000 g/mol. In an example, the weight average molecular weight of the poly(ethylene glycol) linker ranges from about 1,000 g/mol to about 5,000 g/mol. The passivation component 36A is branched when pentaerythritol is used as the linker 40.

Any anchor 44 may be used that can attach to the linker 40 and that can attach to the polymeric hydrogel 32. Example anchors 44 include alkyne-containing functional groups, such as dibenzocyclooctyne (DBCO), bicyclo[6.1.0]nonyne (BCN), and propargyl functional groups, or alkene-containing functional groups, such as norbornene. Another suitable anchor 44 is norbornyl. When the anchor is included, the linker 40 may be described as further including the particular anchor functional group.

Poly(ethylene glycol) may be used as the end group 42 when the linker 40 is poly(meth)acrylate, polyacrylamide, pentaerythritol, and combinations thereof. Examples of the zwitterionic end group are selected from the group consisting of phosphocholine, a sulfobetaine, and a carboxybetaine. Examples of the positively chargeable end group include amine and ammonium. Examples of the anionic polymer end group include polyphosphates, polysulfonates, and polycarboxylates (e.g., poly(meth)acrylic acid). One example of an amphoteric polymer end group is a polymer chain including both carboxylic acid and amine functional groups, such as a co-polymer of methacrylic acid and dimethylammonium ethylmethacrylate.

In some specific examples, the polymeric hydrogel 32 includes terminal azide groups, and the passivation component 36A is selected from the group consisting of dibenzocyclooctyne-poly(ethylene glycol)-carboxylic acid, norbornene-poly(ethylene glycol)-carboxylic acid, bicyclo[6.1.0]non-4-yne-poly(ethylene glycol)-carboxylic acid, dibenzocyclooctyne-poly(ethylene glycol)-amine, norbornene-poly(ethylene glycol)-amine, bicyclo[6.1.0]non-4-yne-poly(ethylene glycol)-amine, dibenzocyclooctyne-poly(ethylene glycol)-hydroxyl, norbornene-poly(ethylene glycol)-hydroxyl, bicyclo[6.1.0]non-4-yne-poly(ethylene glycol)-hydroxyl, dibenzocyclooctyne-poly(ethylene glycol)-sulfonic acid, norbornene-poly(ethylene glycol)-sulfonic acid, bicyclo[6.1.0]non-4-yne-poly(ethylene glycol)-sulfonic acid. In these examples, the dibenzocyclooctyne, norbornene, or bicyclo[6.1.0]non-4-yne is the anchor 44, the poly(ethylene glycol) is the linker 40, and the carboxylic acid, amine, hydroxyl, or sulfonic acid is the end group 42.

In some other specific examples, the polymeric hydrogel 32 includes terminal azide groups; the linker 40 (with the additional anchor 44) is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether; the end group 42 is the zwitterionic end group; and the zwitterionic end group is selected from the group consisting of phosphocholine, a sulfobetaine, and a carboxybetaine.

In still some other specific examples, the polymeric hydrogel 32 includes terminal azide groups; the linker 40 (with the additional anchor 44) is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether; the end group 42 is the amphoteric polymer end group; and the amphoteric polymer end group includes both a carboxylic acid functional group and an amine functional group. One specific example of this amphoteric polymer end group is poly(methacrylic acid-co-dimethylamino ethyl methacrylate).

In yet other examples, the polymeric hydrogel 32 includes terminal azide groups; the linker (with the additional anchor 44) is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether; the end group 42 is the anionic polymer end group; and the anionic polymer end group is poly(meth) acrylic acid.

When the passivation component 36A is the dibenzocyclooctyne-functionalized passivation agent, the dibenzocyclooctyne-functionalized passivation agent is selected from the group consisting of dibenzocyclooctyne-acid, dibenzocyclooctyne-amine, and dibenzocyclooctyne-sulfo-amine. In these examples, the dibenzocyclooctyne is capable of attaching to azides of the polymeric hydrogel 32 and the acid, amine or sulfo-amine is the anti-fouling agent.

The passivation component 36A may be synthesized using suitable polymerization techniques that attach the end group 42 to the linker 40, and in some instances, attach the linker 40 to the anchor 44. As one example, controlled radical polymerization, such as reversible addition fragmentation chain transfer (RAFT) or atom transfer radical polymerization (ATRP), can be used to grow a zwitterionic polymer chain or an amphoteric polymer chain or an anionic polymer chain (examples of the end group 42), followed by end-group modification to introduce the linker 40 and terminal anchor 44. In another example, a thiolated linker and anchor (e.g., DBCO-PEG-SH) may be coupled with 3-[(3-acryloylaminopropyl) dimethylammonio]propanoate or 3-[[2-(acryloyloxy)ethyl]dimethylammonio]propane-1-sulfonate to allow the thiol group to react with the α,β unsaturated carbonyl group via a thiol-acrylate click reaction. In still another example, RAFT polymerization is used to grow the desired linker 40, 40′, and then the trithiocarbonate RAFT end-group may be converted via aminolysis to thiol, followed by the conjugation with DBCO-PEG-maleimide.

In the example of FIG. 1B, the surface chemistry of the polymeric hydrogel 32 may be orthogonal to the surface chemistry of the interstitial regions 30 so that the passivation component 36A attaches to the polymeric hydrogel 32, but not to the interstitial regions 30.

In the example shown in FIG. 1C, the passivation component 36B is attached to at least some of the interstitial regions 30.

The passivation component 36B includes a linker 40′ and an end group 42′, which functions as an anti-fouling agent, attached to one end of the linker 40′. Multiple end groups 42′ may also be attached to side chains or branches of the linker 40′, and thus these particular groups 42′ are not positioned at the opposed terminal ends of the passivation component 36B. The other end of the linker 40′ is capable of attaching to the interstitial regions 30, e.g., through an anchor 44′. The anchor 44′ may be the end group of the linker 40′ or may be a separate molecule that is attached to the linker 40′.

In the example where the passivation component 36B is exclusively attached to the interstitial regions 30, the passivation component 36B includes i) a silane, siloxane, or alkoxy silane small molecule (one example of the linker 40′) with an alkoxy, hydroxyl, carboxylic acid, phosphate, phosphonate, or perfluorinated end group (examples of the end group 40′), or ii) a linker 40′ selected from the group consisting of alkylene, poly(ethylene glycol), poly(meth)acrylic acid, polyacrylamide, poly(2-hydroxyl ethyl (meth)acrylate), and poly(2-hydroxypropyl (meth)acrylamide) and a first end group selected from the group consisting of phosphate, phosphonate, a zwitterionic end group and an amphoteric polymer end group.

Some examples of the passivation component 36B can be synthesized by controlled radical polymerization using reversible addition-fragmentation chain-transfer (RAFT) polymerization or atom transfer radical polymerization (ATRP).

As described herein, some examples of the single layer substrate 18 or the layer 26 of the multi-layer substrate 20 includes or is functionalized to include hydroxyl groups. Silane, siloxane, or alkoxy silane small molecules can react with these hydroxyl groups to attach the alkoxy, hydroxyl, carboxylic acid, or perfluorinated end group 42′ to the interstitial regions 30. Each of these small molecules has a weight average molecular weight ranging from about 100 Daltons to about 1,000 Daltons. Some examples of this type of passivation component 36B include N,N-bis(2-hydroxyethyl)-3-aminopropyltriethoxysilane, bis(methoxyethyl)-3-trimethoxysilylpropylammoniumchloride, carboxyethylsilanetriol disodium salt, and 3-(trihydroxysilyl)-1-propanesulfonic acid. Some examples of the passivation component 36B with the perfluorinated end group are selected from the group consisting of (heptadecafluoro-1,1,2,2-tetrahydrodecyl) trimethoxysilane, 3-(heptafluoroisopropoxy)propyltrimethoxysilane, nonafluorohexyltrimethoxysilane, [perfluoro(polypropyleneoxy)]methoxypropyltrimethoxysilane, 1,3-bis(trifluoropropyl)-1,1,3,3-tetramethyldisilazane, and (tridecafluoro-1,1,2,2-tetrahydrooctyl)dimethylchlorosilane.

In other examples, the passivation component 36B attached to the interstitial regions 30 includes the linker 40′ selected from the group consisting of alkylene, poly(ethylene glycol), poly(meth)acrylic acid, polyacrylamide, poly(2-hydroxyl ethyl (meth)acrylate), and poly(2-hydroxypropyl (meth)acrylamide) and the end group 42′ selected from the group consisting of a zwitterionic end group and an amphoteric polymer end group. In these examples, any examples of the alkylene linker or the poly(ethylene glycol) linker described herein for the linker 40 may be used for the linker 40′, and any examples of the zwitterionic end group and the amphoteric polymer end group described herein for the end group 42 may be used for the end group 42′. These examples of the passivation component 36B may be synthesized using suitable polymerization techniques that attach the end group 42′ to the linker 40′, and in some instances, attach the linker 40′ to the anchor 44′.

In these examples, it is to be understood that any anchor 44′ may be used that can attach to the linker 40′ and that can attach to the interstitial regions 30. Example anchors 44′ include silane, siloxane, or derivatives thereof.

In some specific examples, the linker 40′ is poly(ethylene glycol) and further includes a silane functional group (e.g., anchor 44′) attached to the interstitial regions 30; the end group 42′ is the zwitterionic end group; and the zwitterionic end group is selected from the group consisting of phosphocholine, a sulfobetaine, and a carboxybetaine.

In other specific examples, the linker 40′ is poly(ethylene glycol) and further includes a silane functional group (e.g., anchor 44′) attached to the interstitial regions 30; the end group 42′ is the amphoteric polymer end group; and the amphoteric polymer end group includes both a carboxylic acid functional group and an amine functional group.

In the example of FIG. 1C, the surface chemistry of the interstitial regions 30 may be orthogonal to the surface chemistry of the polymeric hydrogel 32 so that the passivation component 36B attaches to the interstitial regions 30, but not to the polymeric hydrogel 32.

In the example shown in FIG. 1D, the passivation component 36A is attached to the polymeric hydrogel 32 in at least some of the depressions 28 and the passivation component 36B is attached to the interstitial regions 30.

In this example, the respective passivation components 36B, 36A are attached to each of the interstitial regions 30 and to the polymeric hydrogel 32, the respective passivation component 36B attached to the interstitial regions 30 includes i) a silane, siloxane, or alkoxy silane small molecule with an alkoxy, hydroxyl, carboxylic acid, or perfluorinated end group, or ii) a linker 40′ selected from the group consisting of alkylene, poly(ethylene glycol), poly(meth)acrylic acid, polyacrylamide, poly(2-hydroxyl ethyl (meth)acrylate), and poly(2-hydroxypropyl (meth)acrylamide) and an end group 42′ selected from the group consisting of a zwitterionic end group and an amphoteric polymer end group, and the respective passivation component 36A attached to the polymeric hydrogel 30 includes i) a linker 40 selected from the group consisting of alkylene, poly(ethylene glycol), poly(meth)acrylate, polyacrylamide, and pentaerythritol, and combinations thereof, and an end group 42 selected from the group consisting of a poly(ethylene glycol) end group when the linker is poly(meth)acrylate, polyacrylamide, or pentaerythritol, a zwitterionic end group, a hydroxyl end group, a carboxylic acid end group, a sulfonic acid end group, a positively chargeable end group, an alkoxy end group, an anionic polymer end group, and an amphoteric polymer end group, or ii) a dibenzocyclooctyne-functionalized passivation agent.

Any of the examples described herein for the passivation components 36A, 36B may be used in the example shown in FIG. 1C.

In one specific example, the end groups 42, 42′ are each the zwitterionic end group selected from the group consisting of phosphocholine, a sulfobetaine, and a carboxybetaine; the linker 40′ is poly(ethylene glycol) and further includes a silane functional group (e.g., anchor 44′) attached to the interstitial regions 30; and the linker 40 (in some instances, with the anchor 44) is poly(ethylene glycol)-dibenzocyclooctyne, poly(ethylene glycol)-norbornene, bicyclo[6.1.0]non-4-yne-poly(ethylene glycol), bicyclo[6.1.0]non-4-yne-poly(ethylene glycol) methyl ether, or a combination of poly(ethylene glycol) and pentaerythritol.

In another specific example, the end groups 42, 42′ are each the amphoteric polymer end group; the amphoteric polymer end group includes both a carboxylic acid functional group and an amine functional group; the linker 40′ is poly(ethylene glycol) and further includes a silane functional group (e.g., anchor 44′) attached to the interstitial regions 30; and the linker 40 (in some instances, with the anchor 44) is poly(ethylene glycol)-dibenzocyclooctyne or poly(ethylene glycol)-norbornene, bicyclo[6.1.0]non-4-yne-poly(ethylene glycol), bicyclo[6.1.0]non-4-yne-poly(ethylene glycol) methyl ether, or a combination of poly(ethylene glycol) and pentaerythritol.

In the example architecture shown in FIG. 1B, a reversible passivation component 36C may be used instead of the passivation component 36A described herein. In this example, the flow cell 10 includes the substrate 18 or 20 including depressions 28 separated by interstitial regions 30; a polymeric hydrogel 32 positioned within each of the depressions 28; a primer set 34A, 34B attached to the polymeric hydrogel 32; and the reversible passivation component 34C attached to the polymeric hydrogel 32, the reversible passivation component 34C including a lower critical solution temperature polymer or an upper critical solution temperature polymer or a pH responsive monomer co-polymerized with an anti-fouling monomer, or an anti-fouling upper critical solution temperature polymer.

Any example of the substrate 18, 20, the polymeric hydrogel 32, and the primers 34A, 34B may be used in this example of the flow cell 10.

The reversible passivation component 34C includes a lower critical solution temperature (LCST) polymer or an upper critical solution temperature (UCST) polymer or a pH responsive monomer co-polymerized with an anti-fouling monomer, or an anti-fouling upper critical solution temperature polymer. In the former examples, the anti-fouling monomer imparts the anti-fouling behaviour to the reversible passivation component 34C. In the latter examples, the anti-fouling UCST polymer itself exhibits the anti-fouling behaviour.

The LCST polymer or UCST polymer or pH responsive monomer and the anti-fouling monomers can be co-polymerized using controlled radical polymerization techniques.

Because many anti-fouling monomers (e.g., poly(ethylene glycol) methacrylate (PEGMA), zwitterionic monomers, etc.) are hydrophilic, the co-polymerization of these monomers with the LCST polymer or UCST polymer will shift the transition temperature of the LCST polymer or UCST polymer to a higher temperature. As such, in the examples set forth herein, the molar ratio of the anti-fouling monomer to the LCST or UCST may be up to 1:5 (i.e., 20 mol % of the anti-fouling monomer). In one specific example, the molar ratio of the anti-fouling monomer to the LCST polymer or UCST polymer may range from about 1:100 (i.e., 1 mol % of the anti-fouling monomer) to about 1:10 (i.e., 10 mol % of the anti-fouling monomer). Above the maximum molar ratio, the UCST polymer and LCST polymer become thermally non-responsive.

Due to the hydrophilicity of the anti-fouling monomers (e.g., poly(ethylene glycol) methacrylate (PEGMA), zwitterionic monomers, etc.), at least 50 mol % of the pH responsive monomer is co-polymerized with less than 50 mol % of the anti-fouling monomer. This will ensure that the hydrophobic character of the co-polymer is more dominant when the pH is adjusted (up or down depending upon the pH responsive monomer that is used), resulting in the collapse of the reversible passivation component 34C. As such, when the reversible passivation component includes the pH responsive monomer, the molar content of the pH responsive monomer in the reversible passivation component is greater than 50%. In an example, the pH responsive monomer makes from at least 50 mol % to about 80 mol % of the co-polymer and the anti-fouling monomer makes up from about 20 mol % to less than 50 mol % of the co-polymer.

The amount of the anti-fouling monomer may also be determined by its weight average molecular weight. The weight average molecular weight of the anti-fouling monomer in examples of the reversible passivation component 34C that include the anti-fouling monomer may range from about 2,000 g/mol to about 100,000 g/mol. Other examples of the weight average molecular weight of the anti-fouling monomer in examples of the reversible passivation component 34C that include the anti-fouling monomer may range from about 2000 g/mol to about 10,000 g/mol, or from about 10,000 g/mol to about 50,000 g/mol, or from about 50,000 g/mol to about 100,000 g/mol. The molecular weight may affect the mol % of the anti-fouling monomer that is used in order to be within the molar ratio or mol % disclosed herein for the co-polymers.

The LCST or UCST polymer and the anti-fouling UCST polymer each exhibit thermo-responsive properties. These properties enable the reversible passivation component 34C to be activated to exhibit the anti-fouling property or deactivated to suppress the anti-fouling property. In particular, the transition between the hydrophilic, anti-fouling state and the hydrophobic, suppressed anti-fouling state can be controlled through the critical solution temperature. With the LCST polymer, an increase in the temperature above the LCST collapses the co-polymer and suppresses the anti-fouling property. In contrast, a decrease in the temperature to below the LCST renders the co-polymer more hydrophilic, which expands the co-polymer causing it to exhibit the anti-fouling property. An example of the lower critical solution temperature polymer is poly(N-isopropylacrylamide). With the UCST polymer or the anti-fouling UCST polymer, a decrease in the temperature below the UCST collapses the co-polymer and suppresses the anti-fouling property. In contrast, an increase in the temperature to above the UCST renders the co-polymer more hydrophilic, which expands the co-polymer causing it to exhibit the anti-fouling property. An example of the upper critical solution temperature polymer that can be co-polymerized with the anti-fouling monomer includes a co-polymer of acrylamide and acrylonitrile. Examples of the anti-fouling upper critical solution temperature polymer (which exhibits anti-fouling behaviour without being co-polymerized with another anti-fouling monomer) are selected from the group consisting of poly(N-acryloyl glycinamide) and poly(sulfobetaine methacrylate).

The pH responsive monomer exhibits pH-responsive properties. These properties enable the reversible passivation component 34C to be activated to exhibit the anti-fouling property or deactivated to suppress the anti-fouling property. In particular, the transition between the hydrophilic, anti-fouling state and the hydrophobic, suppressed anti-fouling state can be controlled through pH. With pH responsive monomers that are hydrophilic at a higher pH, an increase in the pH to greater than 8 expands the co-polymer causing it to exhibit the anti-fouling property. In contrast, with pH responsive monomers that are hydrophilic at a lower pH, a decrease in the pH to less than 8 expands the co-polymer causing it to exhibit the anti-fouling property.

Examples of pH responsive monomers that are hydrophilic at a pH greater than 8 include a functional group selected from the group consisting of a carboxylic acid group, a phosphoric acid group, a sulfonic acid group, a boronic acid group, and an amino acid group. At the higher pH, the pH responsive segment of the co-polymer (which is formed of this type of pH responsive monomer) becomes deprotonated and hydrophilic, thus activating the anti-fouling segment of the co-polymer (which is formed of the anti-fouling monomer). Examples of pH responsive monomers with a carboxylic acid group include acrylic acid, methacrylic acid, ethylacrylic acid, propylacrylic acid, 4-vinylbenzoic acid, and itaconic acid. Examples of pH responsive monomers with a phosphoric acid group include ethylene glycol acrylate phosphate, ethylene glycol methacrylate phosphate, vinylphosphonic acid, and 4-vinyl-benzyl phosphonic acid. Examples of pH responsive monomers with a sulfonic acid group include vinylsulfonic acid, 4-styrenesulfonic acid, 2-acrylamido-2-methylpropane sulfonic acid, and 3-sulfopropyl methacrylate potassium salt. Examples of pH responsive monomers with a boronic acid group include vinylphenyl boronic acid and 2-acrylamidophenyl boronic acid. Examples of pH responsive monomers with an amino acid group include aspartic acid, L-glutamic acid, and histidine.

Example of pH responsive monomers that are hydrophilic at a pH lower than 8 include a functional group selected from the group consisting of a tertiary amine group, a morpholino group, a pyrrolidine group, a piperazine group, a pyridine group, and an imidazole group. At the lower pH, the pH responsive segment of the co-polymer (which is formed of this type of pH responsive monomer) becomes prontonated and hydrophilic, thus activating the anti-fouling segment of the co-polymer (which is formed of the anti-fouling monomer). Examples of pH responsive monomers with a tertiary amine group include 2-(dimethylamino)ethyl methacrylate, 2-(diethylamino)ethyl methacrylate, 2-(dipropylamino)ethyl methacrylate, 2-(diisopropylamino)ethyl methacrylate, N-[3-(dimethylamino)propyl]methacrylamide, 2-(dimethylamino)ethyl acrylate, 2-(tert-butylamino)ethyl methacrylate, N-N-dialkylvinylbenzylamine (where the alkyl is —CH₃, —CH₂CH₃, or —CH₂CH₂CH₃), and 2-(diethylamino)ethyl methacrylamide. Examples of pH responsive monomers with a morpholino group include 2-(N-morpholinoethyl) methacrylate, 4-acrylooylmorpholine, and 2-(N-morpholinoethyl) methacrylamide. An example of a pH responsive monomer with a pyrrolidine group is N-ethylpyrrolidine methacrylate. An example of a pH responsive monomer with a piperazine group is N-acryloyl-N-alkyl piperazine (where the alkyl is —CH₃, —CH₂CH₃, or —CH₂CH₂CH₃). Examples of pH responsive monomers with a pyridine group include 4-vinylpyridine, and 2-vinylpyridine. Examples of pH responsive monomers with an imidazole group include N-vinylimidazole and 6-(1H-imidazol-1-yl)hexyl-methacrylate.

The anti-fouling monomer is any monomer that can suppress non-specific binding. Examples of anti-fouling monomers include poly(ethylene glycol) (meth)acrylate, phosphocholine (meth)acrylate, sulfobetaine (meth)acrylate, carboxybetaine (meth)acrylate, polyvinylpyrrolidone (PVP), polyoxazolines, polyglycerol, polypeptoids, polyperfluoro alkyls, and polyperfluoro ethers. Specific examples of polyperfluoro alkyls include heptafluorobutyl acrylate, methacrylate or methacrylamide; or trifluoroethyl acrylate, methacrylate, or methacrylamide; or 1H, 1H-perfluorohexyl acrylate, methacrylate, or methacrylamide. Specific examples of polyperfluoro ethers include 1H, 1H-perfluoro-3,6,9-trioxadecyl acrylate, methacrylate, or methacrylamide.

In one example, the reversible passivation component 34C includes the lower critical solution temperature polymer, the lower critical solution temperature polymer is selected from the group consisting of poly(N-isopropylacrylamide) and diethylene glycol methacrylate, and the anti-fouling monomer is selected from the group consisting of poly(ethylene glycol) (meth)acrylate, phosphocholine (meth)acrylate, sulfobetaine (meth)acrylate, and carboxybetaine (meth)acrylate. Two specific examples of the reversible passivation component 34C including an LCST polymer include polyethylene glycol acrylate-co-poly(N-isopropylacrylamide):

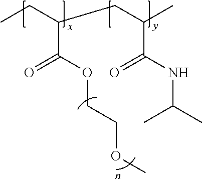

where x is up to 20 mol % and y is up to 80 mol %; and poly(sulfobetaine acrylate)-co-poly(N-isopropylacrylamide):

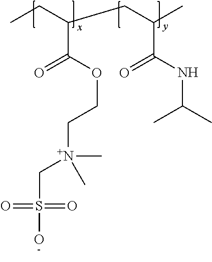

where x is up to 20 mol % and y is up to 80 mol %.

In another example, the reversible passivation component 34C includes the pH responsive monomer; the pH responsive monomer includes a functional group selected from the group consisting of a tertiary amine group, a morpholino group, a pyrrolidine group, a piperazine group, a pyridine group, and an imidazole group; and the anti-fouling monomer is selected from the group consisting of poly(ethylene glycol) (meth)acrylate, phosphocholine (meth)acrylate, sulfobetaine (meth)acrylate, and carboxybetaine (meth)acrylate, polyvinylpyrrolidone (PVP), polyoxazolines, polyglycerol, polypeptoids, polyperfluoro alkyls, and polyperfluoro ethers. In still another example, the reversible passivation component includes the pH responsive monomer; the pH responsive monomer includes a functional group selected from the group consisting of a carboxylic acid group, a phosphoric acid group, a sulfonic acid group, and a boronic acid group; and the anti-fouling monomer is selected from the group consisting of poly(ethylene glycol) (meth)acrylate, phosphocholine (meth)acrylate, sulfobetaine (meth)acrylate, and carboxybetaine (meth)acrylate, polyvinylpyrrolidone (PVP), polyoxazolines, polyglycerol, polypeptoids, polyperfluoro alkyls, and polyperfluoro ethers.

In another example, the reversible passivation component 34C is the upper critical solution temperature polymer, the upper critical solution temperature polymer is a co-polymer of acrylamide and acrylonitrile, and the anti-fouling monomer is selected from the group consisting of poly(ethylene glycol) (meth)acrylate, phosphocholine (meth)acrylate, sulfobetaine (meth)acrylate, and carboxybetaine (meth)acrylate. Two specific examples of the reversible passivation component 34C including an UCST polymer include polyethylene glycol acrylate-co-poly(acrylamide)-co-poly(acrylonitrile):

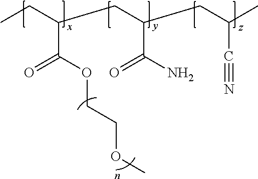

where x is up to 20 mol %, y+z is at least 80 mol %, and 2<n<500 (e.g., PEG acrylate with n ranging from 5-10); and
poly(sulfobetaine acrylate)-co-poly(acrylamide)-co-poly(acrylonitrile):

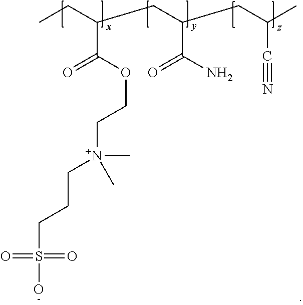

where x is up to 20 mol % and y+z is at least 80 mol % (e.g., where y ranges from 60 mol % to 79 mol % and z ranges from 1 mol % to 20 mol %).

In another example, the reversible passivation component 34C is the anti-fouling upper critical solution temperature polymer, and the anti-fouling upper critical solution temperature polymer is selected from the group consisting of poly(N-acryloyl glycinamide):

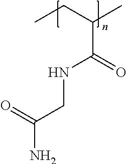

and poly(sulfobetaine methacrylate) (two examples of which include:

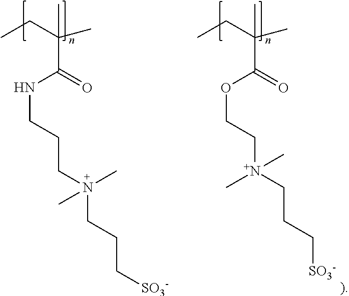

### Method for Making the Flow Cell

Any example of the flow cell 10 may be generated as described herein.

At the outset of the method, the depressions 28 are defined in the single layer substrate 18 or in the layer 26 of the substrate 20. Defining the depressions 28 involves nanoimprint lithography or dry etching.

In one example, nanoimprint lithography is used to define the depressions 28. In this example, a working stamp is pressed into single layer substrate 18 or the layer 26 while the material is soft, which creates an imprint of the working stamp features in the material. In this example, each working stamp feature is a negative replica of the depression 28. The material may then be cured with the working stamp in place.

Curing may be accomplished by exposure to actinic radiation, such as visible light radiation or ultraviolet (UV) radiation, when a radiation-curable resin material is used; or by exposure to heat when a thermal-curable resin material is used. Curing may promote polymerization and/or cross-linking. As an example, curing may include multiple stages, including a softbake (e.g., to drive off any liquid carrier that may be used to deposit the resin) and a hardbake. The softbake may take place at a lower temperature, ranging from about 50° C. to about 150° C. The duration of the hardbake may last from about 5 seconds to about 10 minutes at a temperature ranging from about 100° C. to about 300° C. Examples of devices that can be used for softbaking and/or hardbaking include a hot plate, oven, etc.

After curing, the working stamp is released. This creates topographic features (e.g., the depressions 28) in the single layer substrate 18 or the layer 26.

Dry etching, a combination of gray scale lithography and dry etching, or silicon wet etching may be used to define the depressions 28. With gray scale lithography and dry etching, a photoresist and gray scale photo mask may be used to define the pattern of the depressions 28.

In some instances, the substrate 18 or layer 26 may be activated, e.g., through silanization or plasma ashing. Activation is a process that generates reactive groups at the surface of the single layer substrate 18 or the outermost layer 26 of the multi-layer substrate 20. Activation may be accomplished using silanization or plasma ashing. While the figures do not depict a separate silanized layer or —OH groups from plasma ashing, it is to be understood that activation generates a silanized layer or —OH groups at the surface of the activated single layer substrate 18 or the layer 26 to covalently attach the polymeric hydrogel 32, and in some instances, the passivation component 36B. The activation process is performed if the single layer substrate 18 or the layer 26 does not inherently include the reactive groups, e.g., to covalently attach the polymeric hydrogel 32.

The polymeric hydrogel 32 is then applied over the single layer substrate 18 or the layer 26 such that it aligns (e.g., in conformal with) the depressions 28 and the interstitial regions 30. A curing process may be performed after deposition. The polymeric hydrogel 32 covalently attaches to the single layer substrate 18 or the layer 26. Covalent linking is helpful for maintaining the primers 34A, 34B in the desired regions throughout the lifetime of the flow cell 10 during a variety of uses.

The polymeric hydrogel 32 that overlies the interstitial regions 30 may then be removed via polishing. The polishing process may be performed with a chemical slurry (including, e.g., an abrasive, a buffer, a chelating agent, a surfactant, and/or a dispersant). Alternatively, polishing may be performed with a solution that does not include the abrasive particles.

The chemical slurry may be used in a chemical mechanical polishing system to polish the interstitial regions 30. The polishing head(s)/pad(s) or other polishing tool(s) is/are capable of polishing the polymeric hydrogel 32 that is present over the interstitial regions 30 while leaving the polymeric hydrogel 32 in the depressions 28 at least substantially intact. As an example, the polishing head may be a Strasbaugh ViPRR II polishing head. The polishing process can remove the polymeric hydrogel 32 from the interstitial regions 30 without deleteriously affecting the underlying single layer substrate 18 or layer 26.

Cleaning and drying processes may be performed after polishing. The cleaning process may utilize a water bath and sonication. The water bath may be maintained at a relatively low temperature ranging from about 22° C. to about 30° C. The drying process may involve spin drying, or drying via another suitable technique.

The primers 34A, 34B are then attached to the polymeric hydrogel 32. In some examples, the primers 34A, 34B may be pre-grafted to the polymeric hydrogel 32 before it is applied in the depressions 28. In these examples, additional primer grafting is not performed.

In other examples, the primers 34A, 34B are not pre-grafted to the polymeric hydrogel 32. In these examples, the primers 34A, 34B may be grafted after the polymeric hydrogel 32 is applied. Grafting may be accomplished using any suitable grafting technique. As examples, grafting may be accomplished by flow through deposition (e.g., using a temporarily bound lid), dunk coating, spray coating, puddle dispensing, or by another suitable method. Each of these example techniques may utilize a primer solution or mixture, which includes the primers 34A, 34B, water, a buffer, and a catalyst. With any of the grafting methods, the primers 34A, 34B attach to the reactive groups of the polymeric hydrogel 32, and have no affinity for the interstitial regions 30.

The passivation component 36A or 36C is then attached to the polymeric hydrogel 32, or the passivation component 36B is then attached to the interstitial regions 30, or both the passivation components 36A, 36B are attached to the polymeric hydrogel 32 and the interstitial regions 30. The attachment of the passivation components 36A and/or 36B, or 36C may be performed using any of the grafting techniques at conditions that enable the attachment between the passivation component 36A or 36C and the polymeric hydrogel 32, or the passivation component 36B and the interstitial regions 30, or both the passivation components 36A, 36B and the polymeric hydrogel 32 and the interstitial regions 30. In some examples, copper click chemistry may be used to attach the passivation component 36A or 36C (e.g., including an alkyne) to the polymeric hydrogel 32; or copper free chemistry may be used to attach the passivation component 36A or 36C (e.g., including DBCO, BCN, or norbornene) to the polymeric hydrogel 32; or copper free chemistry may be used to attach the passivation component 36B to the interstitial regions 30. In another example, attaching a perfluorosilane to the interstitial regions 30 (made up of a resin) may involve dissolving a solution of perfluorosilane in a mixture of ethanol/water (˜95%:5%), then drop casting the solution onto the interstitial regions 30 or flowing the solution the flow cell 10, and incubating the solution in contact with the interstitial regions 30 at room temperature for a few hours.

When the passivation components 36A or 36C specifically react with the polymeric hydrogel 32 and not with the interstitial regions 30 or the bonding regions, the interstitial regions 30 and the bonding regions are not masked during the attachment of the passivation components 36A or 36C. When the passivation component 36B specifically reacts with the interstitial regions 30 and with the bonding regions, but not with the polymeric hydrogel 32, the polymeric hydrogel 32 is not masked during the attachment of the passivation component 36B but the bonding regions may be masked to keep the passivation component 36B from binding at the bonding regions.

It is to be understood that the passivation components 36A and/or 36B, or 36C may be attached to the polymeric hydrogel 32 and/or to the interstitial regions 30 in any desired order.

If the flow cell 10 is to be enclosed, the patterned structure 14A, 14B, 14C that is formed can then be bonded to the lid 16 or another patterned structure, e.g., 14B′, using the spacer layer 22 and a suitable bonding method. The lid 16 and the patterned structure 14A, 14B, 14C or two patterned structures, e.g., 14B and 14B′, may be bonded using any suitable technique, such as laser bonding, diffusion bonding, anodic bonding, eutectic bonding, plasma activation bonding, glass frit bonding, or others methods known in the art.

### Methods for Using the Flow Cell

The flow cells 10 disclosed herein may be used in a sequencing operation, such as sequencing-by-synthesis.

The following example describes sequencing-by-synthesis when the flow cell 10 includes the passivation component 36A and/or 36B.

Sequencing-by-synthesis utilizes a plurality of library templates (i.e., template nucleic acid strands). The library templates may be prepared from any nucleic acid sample (e.g., a DNA sample or an RNA sample). The DNA nucleic acid sample may be fragmented into single-stranded, similarly sized (e.g., <1000 base pairs (bp)) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 34A, 34B in the depressions 28. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to a suspension, which includes a liquid carrier (e.g., water and a buffer). The library template suspension is introduced into the flow cell 10, where they are hybridized, for example, to one of two types of primers 34A, 34B or the capture primer immobilized within each depression 28.

Amplification of the template nucleic acid strand(s) within each depression 28 may be initiated to form a cluster of the template strands. In one example, amplification involves cluster generation. In one example of cluster generation, the library templates are copied from the hybridized primers by 3′ extension using a high-fidelity DNA polymerase. The original library templates are denatured, leaving the copies immobilized all around the depression 28. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters of amplicons within the depressions 28. Each cluster of double stranded bridges is denatured. In an example, the reverse strands are removed by specific base cleavage, leaving forward template strands. This example of clustering is referred to as bridge amplification, and is one example of the amplification that may be performed. It is to be understood that other amplification techniques may be used.

Sequencing primers may then be introduced to the flow cell 10. The sequencing primers hybridize to a complementary portion of the sequence of the template strands that are attached within the depressions 28. These sequencing primers render the template strands ready for sequencing.

An incorporation mix including labeled nucleotides may then be introduced into the flow cell 10, e.g., via an inlet that leads to the flow channel 12. In addition to the labeled nucleotides, the incorporation mix may include water, a buffer, and enzymes (e.g., polymerases) capable of nucleotide incorporation. When the incorporation mix is introduced into the flow cell 10, the mix enters the flow channel 12, and contacts the anchored and sequence ready template strands.

The incorporation mix is allowed to incubate in the flow cell 10, and labeled nucleotides (including optical labels) are incorporated by respective polymerases into the nascent strands along the template strands/amplicons in the depressions 28. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the template strand. Incorporation occurs in at least some of the template strands across the pre-clustered nanostructure during a single sequencing cycle.

In some examples of the flow cell 10, the presence of the passivation component(s) 36A and/or 36B at least reduces the non-specific binding of the labeled nucleotides, polymerase(s), etc. on the polymeric hydrogel 32 and/or the interstitial regions 30.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 10 during a wash cycle. The wash cycle may involve a flow-through technique, where a washing solution (e.g., buffer) is directed into, through, and then out of flow channel 12, e.g., by a pump or other suitable mechanism. This wash cycles removes the non-incorporated labeled nucleotides, polymerase(s), etc. from the flow cell 10.

Without further incorporation taking place, the most recently incorporated labeled nucleotides can be detected through an imaging event. During the imaging event, an illumination system may provide an excitation light to the flow cell 10. The optical labels of the incorporated labeled nucleotides emit optical signals in response to the excitation light. Because non-specific binding is reduced or eliminated, the signal to noise ratio during the imaging event is improved.

After imaging is performed, a cleavage mix may then be introduced into the flow cell 10. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strands are sequenced.

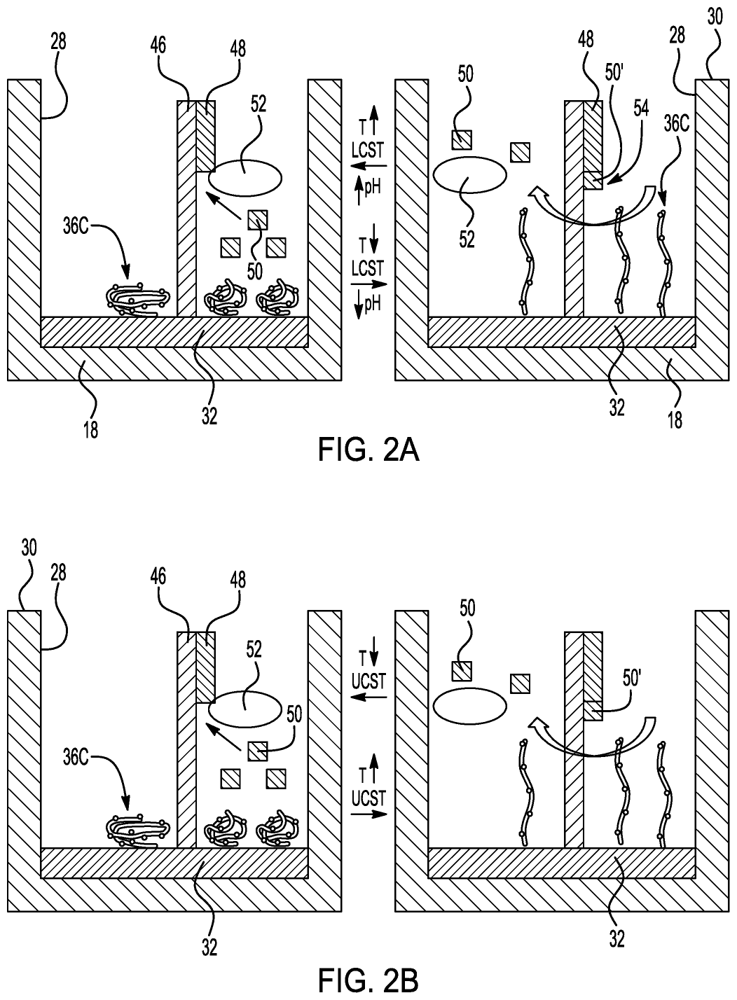

Sequencing-by-synthesis is slightly different when different examples of the passivation component 36C are utilized. The description of FIG. 2A relates to sequencing-by-synthesis when the flow cell 10 includes the passivation component 36C with an LCST polymer or with a co-polymer of the anti-fouling monomer and one type of pH responsive monomer, and the description of FIG. 2B relates to sequencing-by-synthesis when the flow cell 10 includes the passivation component 36C with a UCST polymer or with a co-polymer of the anti-fouling monomer and another type of pH responsive monomer.

In either example, library template preparation may be performed in the same manner as described herein. The library templates are introduced into the flow cell 10 and cluster generation may be performed in the same manner as described herein. The sequencing primers may then be introduced to render the template strands ready for sequencing. Each of FIG. 2A and FIG. 2B illustrates a portion of a depression 28 with the polymeric hydrogel 32 after cluster generation has been performed and sequencing primers have been introduced. For ease of illustration, a single template strand (amplicon) 46 and a single sequencing primer 48 hybridized to the template strand 46 are depicted.

When the passivation component 36C includes the lower critical solution temperature (LCST) polymer co-polymerized with the anti-fouling monomer, the method includes incorporating the incorporation mix, which includes labeled nucleotides 50 and the incorporation enzyme 52 (e.g., a polymerase) into the flow cell 10 (which includes the reversible passivation component 36C attached to the polymeric hydrogel 32); increasing a temperature of the flow cell 10 at or above a lower critical solution temperature of the LCST polymer to collapse the reversible passivation component 36C (left side of FIG. 2A) and to initiate a sequencing cycle in the flow cell 10; and after the sequencing cycle, lowering the temperature of the flow cell below the lower critical solution temperature of the LCST polymer, thereby expanding the reversible passivation component 36C (right side of FIG. 2A); and flowing a washing solution through the flow cell 10 while the reversible passivation component 36C is expanded.

In this example method, the temperature to which the flow cell 10 is increased is at or above the lower critical solution temperature of the LCST polymer that is used in the reversible passivation component 36C. As such, the temperature will depend upon the LCST polymer that is used. As an example when poly(N-isopropylacrylamide) is used as the LCST polymer in the reversible passivation component 36C, the LCST ranges from about 22° C. to about 42° C. depending upon the molar ratio of N-isopropylacrylamide in the co-polymer that makes up the reversible passivation component 36C. Above the LCST, the reversible passivation component 36C is more hydrophobic and transitions to its collapsed state. In this state, the reversible passivation component 36C does not disrupt or interfere with the incorporation of the labeled nucleotide 50 into the nascent strand 54 that is formed along the template strand 46, and thus the anti-fouling property is suppressed.

The temperature to which the flow cell 10 is increased may also be suitable for the incorporation reaction to take place. In these examples, the temperature is raised to from about 50° C. to about 70° C. In one example, the temperature is raised to about 65° C.

The incorporated labeled nucleotide 50′ (shown on the right side of FIG. 2A) may include a reversible termination property due to the presence of a 3′ OH blocking group, which terminates further sequencing primer extension once the labeled nucleotide 50 has been added to the growing nascent strand 54. The imaging event may then be performed.

The incorporation and imaging of the labeled nucleotide 50′ equates to a sequencing cycle, and temperature of the flow cell 10 is then lowered to below the lower critical solution temperature of the LCST polymer. This renders the reversible passivation component 36C more hydrophilic, and thus it expands as shown on the right side of FIG. 2A. In the expanded state, the reversible passivation component 36C is anti-fouling as it creates a barrier between the polymeric hydrogel 32 and the non-incorporated labeled nucleotides 50 and enzymes 52 in the incorporation mix.

A washing solution is introduced into the flow cell 10 while the reversible passivation component 36C is expanded. The flow of the washing solution from the inlet to the outlet removes the non-incorporated labeled nucleotides 50 and the enzymes 52.

Another sequencing cycle with the transition of the reversible passivation component 36C between states may then be performed.

When the passivation component 36C includes the upper critical solution temperature (UCST) polymer co-polymerized with the anti-fouling monomer, the method includes incorporating the incorporation mix, which includes labeled nucleotides 50 and the incorporation enzyme 52 (e.g., a polymerase) into the flow cell 10 (which includes the reversible passivation component 36C attached to the polymeric hydrogel 32); increasing a temperature of the flow cell 10 below an upper critical solution temperature of the UCST polymer to collapse the reversible passivation component 36C (left side of FIG. 2B) and to initiate a sequencing cycle in the flow cell 10; and after the sequencing cycle, increasing the temperature of the flow cell 10 above the upper critical solution temperature of the LCST polymer, thereby expanding the reversible passivation component 36C (right side of FIG. 2B); and flowing a washing solution through the flow cell 10 while the reversible passivation component 36C is expanded.

In this example method, the temperature to which the flow cell 10 is increased is below the upper critical solution temperature of the UCST polymer that is used in the reversible passivation component 36C. As such, the temperature will depend upon the UCST polymer that is used. As an example when poly(acrylamide)-co-poly(acrylonitrile) is used as the UCST polymer in the reversible passivation component 36C, the UCST ranges from about 20° C. to about 70° C. depending upon the molar ratio of the poly(acrylamide)-co-poly(acrylonitrile) in the co-polymer that makes up the reversible passivation component 36C. Below the UCST, the reversible passivation component 36C is more hydrophobic and transitions to its collapsed state. In this state, the reversible passivation component 36C does not disrupt or interfere with the incorporation of the labeled nucleotide 50 into the nascent strand 54 that is formed along the template strand 46, and thus the anti-fouling property is suppressed.

The temperature to which the flow cell 10 is increased may also be suitable for the incorporation reaction to take place. In these examples, the temperature is raised to from about 50° C. to about 70° C. (e.g., about 65° C.), which is less than the UCST of the reversible passivation component 36C.

The incorporated labeled nucleotide 50′ (shown on the right side of FIG. 2B) may include a reversible termination property due to the presence of a 3′ OH blocking group, which terminates further sequencing primer extension once the labeled nucleotide 50 has been added to the growing nascent strand 54. The imaging event may then be performed.

The incorporation and imaging of the labeled nucleotide 50′ equates to a sequencing cycle, and temperature of the flow cell 10 is then increased to above the upper critical solution temperature of the UCST polymer. This renders the reversible passivation component 36C more hydrophilic, and thus it expands as shown on the right side of FIG. 2B. In the expanded state, the reversible passivation component 36C is anti-fouling as it creates a barrier between the polymeric hydrogel 32 and the non-incorporated labeled nucleotides 50 and enzymes 52 in the incorporation mix.

A washing solution is introduced into the flow cell 10 while the reversible passivation component 36C is expanded. The flow of the washing solution from the inlet to the outlet removes the non-incorporated labeled nucleotides 50 and the enzymes 52.

Another sequencing cycle with the transition of the reversible passivation component 36C between states may then be performed.

When the passivation component 36C includes the pH responsive monomer co-polymerized with the anti-fouling monomer, the method includes incorporating the incorporation mix, which includes labeled nucleotides 50 and the incorporation enzyme 52 (e.g., a polymerase) into the flow cell 10 (which includes the reversible passivation component 36C attached to the polymeric hydrogel 32); increasing a pH within the flow cell 10 to at least 8.0 to deactivate and collapse the pH responsive monomer and to initiate a sequencing cycle in the flow cell 10 (left side of FIG. 2A); and after the sequencing cycle, lowering the pH within the flow cell 10 to between at least 7.0 and to 9.0 or less, thereby expanding the reversible passivation component 36C (right side of FIG. 2A); and flowing a washing solution through the flow cell 10 while the reversible passivation component 36C is expanded.

In this example, it is to be understood that the pH responsive monomer is selected from any of the examples set forth herein, so long as the monomer is more hydrophilic when exposed to the increased pH and more hydrophobic when exposed to the decreased pH. The monomer selected will thus be dependent upon the various pH values that are to be used in the method.

In this example method, the pH within the flow cell 10 is increased to at least 8.0. The pH used will depend upon the pKA of the pH responsive monomer that is included in the co-polymer. At the higher pH, the reversible passivation component 36C will deprotonate and become more hydrophobic, causing it to transition to its collapsed state. In this state, the reversible passivation component 36C does not disrupt or interfere with the incorporation of the labeled nucleotide 50 into the nascent strand 54 that is formed along the template strand 46, and thus the anti-fouling property is suppressed.

The increased pH may also be suitable for the incorporation reaction to take place. In one example, the increased pH ranges from 8.0 to 10.0. In some examples, the increased pH ranges from 9.0 to 10.0.

The incorporated labeled nucleotide 50′ (shown on the right side of FIG. 2A) may include a reversible termination property due to the presence of a 3′ OH blocking group, which terminates further sequencing primer extension once the labeled nucleotide 50 has been added to the growing nascent strand 54. The imaging event may then be performed.

The incorporation and imaging of the labeled nucleotide 50′ equates to a sequencing cycle, and the pH within the flow cell 10 is then lowered to protonate the pH responsive monomer(s) of the co-polymer. This renders the reversible passivation component 36C more hydrophilic, and thus it expands as shown on the right side of FIG. 2A. In the expanded state, the reversible passivation component 36C is anti-fouling as it creates a barrier between the polymeric hydrogel 32 and the non-incorporated labeled nucleotides 50 and enzymes 52 in the incorporation mix.

A washing solution is introduced into the flow cell 10 while the reversible passivation component 36C is expanded. The flow of the washing solution from the inlet to the outlet removes the non-incorporated labeled nucleotides 50 and the enzymes 52. The pH of the washing solution may range from 7.0 to 9.0, which keeps the selected pH responsive monomer, and thus the co-polymer, in the expanded state.

Another sequencing cycle with the transition of the reversible passivation component 36C between states may then be performed.

In another example method, the pH responsive monomer may be selected to be more hydrophilic at a lower pH (e.g., 7.5). Scanning and imaging processes during sequencing may be conducted at the lower pH, and the anti-fouling effect (expanded state) of the pH responsive monomer may be desirable for preventing false fluorescent signals cause by non-specific binding. In some instances, the pH responsive monomer may be selected to be more hydrophilic at a pH ranging from about 7.0 to about 9.0 so that it is expanded during both washing (as described in reference to FIG. 2A) and during scanning and imaging.

### Additional Methods for Reducing Non-Specific Binding

The flow cells 10 disclosed herein reduce non-specific binding by including the passivation component(s) 36A, 36B, 36C. These flow cells 10 can also be used with a wash solution that reduces non-specific binding. It is to be understood that this wash solution can alternatively be used with flow cells 10 that include the substrate 18, 20 with the depressions 28 defined therein, the polymeric hydrogel 32 in the depressions 28, and the primers 34A, 34B attached to the polymeric hydrogel 32, but do not include the passivation components 36A, 36B, 36C.

The wash solution is an aqueous solution including water and a cyclodextrin additive, alone or in combination with a buffer agent, a salt, a surfactant, a biocide, a chelating agent, a protein, an enzyme, a protein blocker, or combinations thereof.

The cyclodextrin additive is selected from the group consisting of β-cyclodextrin, β-cyclodextrin sulfated sodium salt, 2-hydroxypropyl β-cyclodextrin, succinyl-β-cyclodextrin, γ-cyclodextrin, and combinations thereof. These charged cyclodextrin additives reduce non-specific binding that can take place during the sequencing operation.

A suitable buffer agent is the Tris buffer. Suitable salts include sodium chloride, sodium citrate, or the like. A suitable surfactant includes TWEEN polysorbates (e.g., TWEEN 20). A suitable biocide includes ProClin™ (available through MilliporeSigma). A suitable chelating agent is ethylenediaminetetraacetic acid (EDTA). A suitable protein includes proteinase K (PrK), which can function as an active reagent in the wash solution. A suitable enzyme includes a polymerase, such as Pol 1901. Bovine serum albumin is a reagent that serves as a protein blocker. Any of the additives may be included alone or in combination with the cyclodextrin additive to further reduce non-specific binding.

In one example, the washing solution includes water, the salt at a concentration ranging from about 25 mM to about 50 mM, the surfactant in an amount ranging from about 0.01 wt % to about 0.1 wt %, the cyclodextrin additive in an amount ranging from about 1 wt % to about 10 wt %, and optionally the buffer agent, the biocide, and/or the chelating agent. The washing solution may have a relatively high pH, e.g., ranging from about 7 to about 10.

A method for reducing non-specific binding using an example of the washing solution includes performing a sequencing cycle in a flow cell 10 (or a flow cell that does not include the passivation component 36A, 36B, 36C) with an incorporation mix that includes an enzyme 52 and a labeled nucleotide 50; and after the sequencing cycle, performing a wash cycle in the flow cell 10 with an example of the wash solution described herein that includes the cyclodextrin additive.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

NEXTSEQ® flow cells from Illumina Inc. were used in this example. The flow cells included PAZAM in the depressions with one of the P5 primers and one of the P7 primers grafted thereto. One NEXTSEQ® flow cell was used as a control and no passivation components were added. Three other NEXTSEQ® flow cells were altered with passivation components, namely mPEG-DBCO. Three different molecular weight mPEG moieties were used, namely 1,000 Daltons, 2,000 Daltons, and 5,000 Daltons.

Solutions of the mPEG-DBCO (0.2 mM) were respectively introduced into the flow cells, and the DBCO (anchor/linker) was allowed to react with the PAZAM to passivate the depressions with the mPEG moieties (anti-fouling or passivating agent). This reaction involved a copper free click chemistry reaction. The solution of mPEG-DBCO was prepared with 1M sodium sulfate, was introduced into the flow cell, and was allowed to incubate on the flow cell surface for about 2 hours at 60° C., during which the DBCO functional group reacted with the azide on the PAZAM.

A Typhoon imager was used to measure the fluorescence intensity after passivation. This provided baseline values.

Each flow cell was then incubated (for about 2 hours at 60° C.) with an incorporation mix containing a polymerase (Pol 1901) and labeled nucleotides (red/green nucleotides used with the NEXTSEQ™ 500 platform). After incubation, a cleavage mix was introduced into and removed from each flow cell.

The Typhoon imager was used again to measure the fluorescence intensity after incubation and cleavage mix flow through. At this point, a lower intensity compared to the baseline means that there are less labeled nucleotides sticking on the PAZAM surface, indicating that the passivating agents have helped reduce non-specific binding.

Each flow cell was then washed with a wash buffer solution including water, Tris buffer, sodium chloride, EDTA, TWEEN™ 20, and PROCLIN™.

The Typhoon imager was used again to measure the fluorescence intensity after washing. At this point, a lower intensity compared to the results after incubation further supports that there are less labeled nucleotides sticking on the PAZAM surface, indicating that the passivating agents have helped reduce non-specific binding.

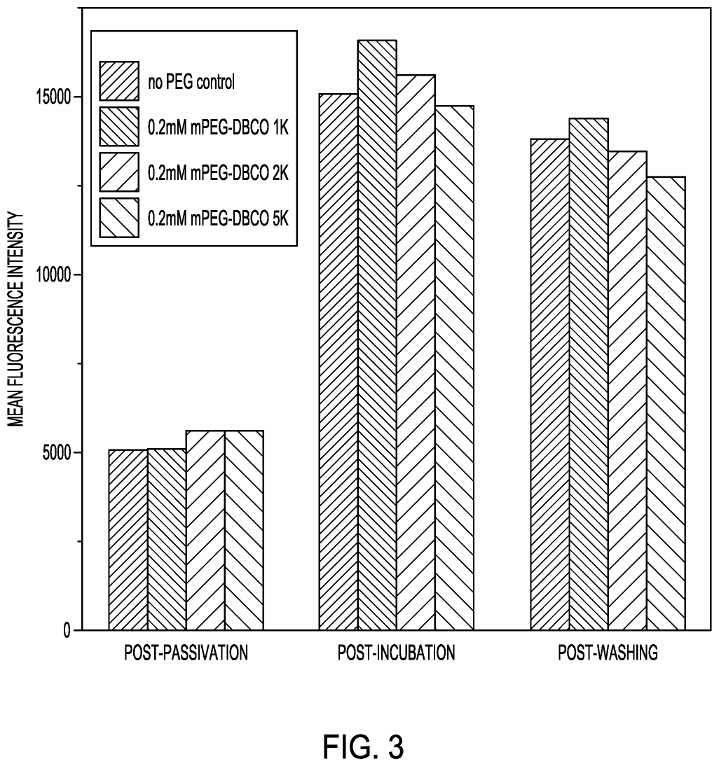

The fluorescence intensity results after passivation, incubation (and cleavage mix flow through), and washing are shown in FIG. 3. These results illustrate that DBCO is a suitable anchor for the passivation component and that PEG is a suitable passivating agent. The 5,000 Dalton PEG exhibited a 15% improvement in non-specific binding compared to the control.

### Example 2

NEXTSEQ® 500 flow cells from Illumina Inc. were used in this example. The flow cells included PAZAM in the depressions with one of the P5 primers and one of the P7 primers grafted thereto.

In the first experiment, four flow cells did not have any passivation components added thereto and four flow cells were altered with passivation components, namely mPEG (5,000 Daltons)-DBCO.

Solutions of the mPEG-DBCO (0.5 mM) were respectively introduced into the four flow cells, and the DBCO (anchor/linker) was allowed to react with the PAZAM to passivate the depressions with the mPEG moieties (anti-fouling or passivating agent). Passivation was performed as described in Example 1.

A Typhoon imager was used to measure the fluorescence intensity of the non-passivated flow cells and the passivated flow cells. This provided baseline values.

Each flow cell was then incubated with an incorporation mix followed by introduction and removal of a cleavage mix as described in Example 1.

The Typhoon imager was used again to measure the fluorescence intensity after incubation and cleavage mix flow through. For the PEG-passivated flow cells, a lower intensity compared to the non-PEG-passivated flow cells means that there are less labeled nucleotides sticking on the passivated PAZAM surface, indicating that the passivating agents have helped reduce non-specific binding.

Each flow cell was then washed with a different wash solution. Each wash solution included water, Tris buffer, sodium chloride, EDTA, TWEEN™ 20, and PROCLIN™. A control solution included no cyclodextrin additive. Three of the wash solutions included different amounts of 2-hydroxypropyl β-cyclodextrin (1%, 5%, and 10%).

The Typhoon imager was used again to measure the fluorescence intensity after washing. At this point, a lower intensity compared to the results after incubation indicates that the wash solution has helped reduce non-specific binding.

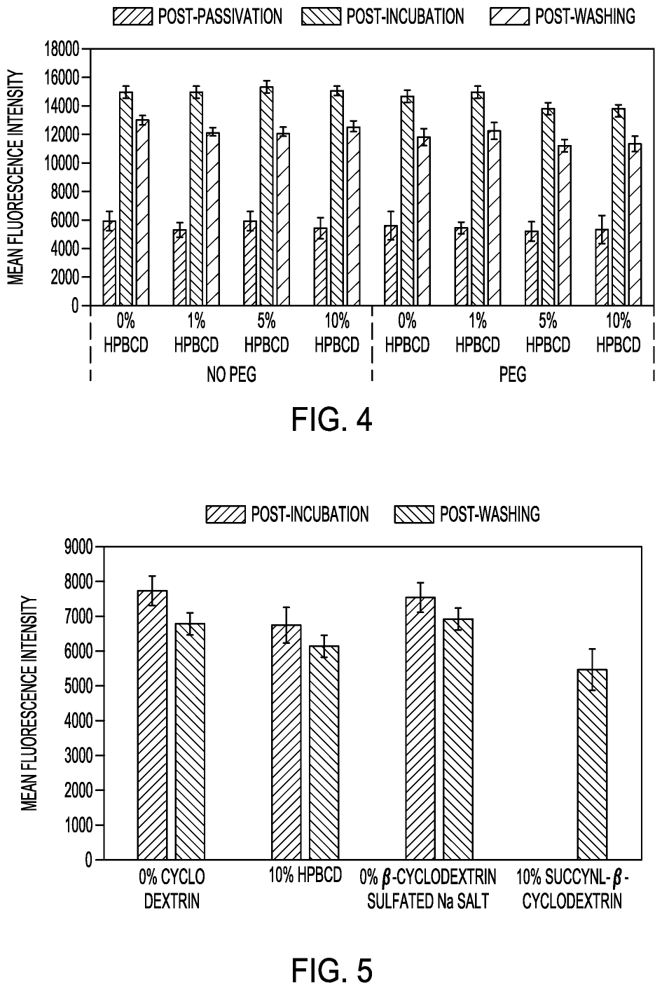

The fluorescence intensity results after passivation, incubation (and cleavage mix flow through), and washing are shown in FIG. 4. These results illustrate that from 5% to 10% of the cyclodextrin in the washing solution slightly reduces non-specific binding. The combination of the 5,000 Dalton PEG and the cyclodextrin containing wash solution exhibited the biggest improvement in non-specific binding compared to the control examples washed without any cyclodextrin in the wash solution.

The 10% 2-hydroxypropyl β-cyclodextrin wash solution was then compared with a control wash solution (all of the noted components but no cyclodextrin) and two other wash solutions containing all of the noted components and two different cyclodextrins. One of the wash solutions contained 10% β-cyclodextrin sulfated sodium salt and the other of the wash solutions contained 10% succinyl-β-cyclodextrin.

In this second experiment, four flow cells were altered with passivation components, namely mPEG (5,000 Daltons)-DBCO.

Solutions of the mPEG-DBCO (0.2 mM) were respectively introduced into the four flow cells, and the DBCO (anchor/linker) was allowed to react with the PAZAM to passivate the depressions with the mPEG moieties (anti-fouling or passivating agent). Passivation was performed as described in Example 1. Each flow cell was then incubated with an incorporation mix followed by introduction and removal of a cleavage mix as described in Example 1.

A Typhoon imager was used to measure the fluorescence intensity after incubation and cleavage mix flow through. For the PEG-passivated flow cells, a lower intensity compared to the non-PEG-passivated flow cell means that there are less labeled nucleotides sticking on the passivated PAZAM surface, indicating that the passivating agents have helped reduce non-specific binding.

Each flow cell was then washed with the wash solutions described above with no cyclodextrin, or 10% of one of 2-hydroxypropyl β-cyclodextrin, or β-cyclodextrin sulfated sodium salt, or succinyl-β-cyclodextrin.

The Typhoon imager was used again to measure the fluorescence intensity after washing. At this point, a lower intensity compared to the results after incubation indicates that the wash solution has helped reduce non-specific binding.

The fluorescence intensity results after passivation, incubation (and cleavage mix flow through), and washing are shown in FIG. 5. The wash solutions containing 10% 2-hydroxypropyl β-cyclodextrin and 10% succinyl-β-cyclodextrin were particularly suitable reducing non-specific binding.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

1. A flow cell, comprising:
a substrate including depressions separated by interstitial regions;
a polymeric hydrogel positioned within each of the depressions;
a primer set attached to the polymeric hydrogel; and
one of: a passivation component attached to the interstitial regions, a passivation component the polymeric hydrogel, or respective passivation components attached to each of the interstitial regions and the polymeric hydrogel, wherein:
the passivation component is exclusively attached to the interstitial regions and includes i) a silane, siloxane, or alkoxy silane small molecule with an alkoxy, hydroxyl, carboxylic acid, or perfluorinated end group, or ii) a first linker selected from the group consisting of alkylene, poly(ethylene glycol), poly(meth)acrylic acid, polyacrylamide, poly(2-hydroxyl ethyl (meth)acrylate), and poly(2-hydroxypropyl (meth)acrylamide) and a first end group selected from the group consisting of phosphate, phosphonate, a zwitterionic end group and an amphoteric polymer end group; or
the passivation component is exclusively attached to the polymeric hydrogel and includes i) a second linker selected from the group consisting of alkylene, poly(ethylene glycol), poly(meth)acrylate, polyacrylamide, pentaerythritol, and combinations thereof, and a second end group selected from the group consisting of a poly(ethylene glycol) end group when the linker is poly(meth)acrylate, polyacrylamide, pentaerythritol, and combinations thereof, a zwitterionic end group, a hydroxyl end group, a carboxylic acid end group, a sulfonic acid end group, a positively chargeable end group, an alkoxy end group, an anionic polymer end group, and an amphoteric polymer end group, or ii) a dibenzocyclooctyne-functionalized passivation agent; or
the respective passivation components are attached to each of the interstitial regions and to the polymeric hydrogel, the respective passivation component attached to the interstitial regions includes i) a silane, siloxane, or alkoxy silane small molecule with an alkoxy, hydroxyl, carboxylic acid, or perfluorinated end group, or ii) a third linker selected from the group consisting of alkylene, poly(ethylene glycol), poly(meth)acrylic acid, polyacrylamide, poly(2-hydroxyl ethyl (meth)acrylate), and poly(2-hydroxypropyl (meth)acrylamide) and a third end group selected from the group consisting of a zwitterionic end group and an amphoteric polymer end group, and the respective passivation component attached to the polymeric hydrogel includes i) a fourth linker selected from the group consisting of alkylene, poly(ethylene glycol), poly(meth)acrylate, polyacrylamide, and pentaerythritol, and combinations thereof, and a fourth end group selected from the group consisting of a poly(ethylene glycol) end group when the linker is poly(meth)acrylate, polyacrylamide, or pentaerythritol, a zwitterionic end group, a hydroxyl end group, a carboxylic acid end group, a sulfonic acid end group, a positively chargeable end group, an alkoxy end group, an anionic polymer end group, and an amphoteric polymer end group, or ii) a dibenzocyclooctyne-functionalized passivation agent.

2. The flow cell as defined in claim 1, wherein:
the passivation component is exclusively attached to the polymeric hydrogel; and
the second linker further includes an alkyne-containing functional group or an alkene-containing functional group attached to the polymeric hydrogel.

3. The flow cell as defined in claim 2, wherein:
the polymeric hydrogel includes terminal azide groups; and
the passivation component is selected from the group consisting of dibenzocyclooctyne-poly(ethylene glycol)-carboxylic acid, norbornene-poly(ethylene glycol)-carboxylic acid, bicyclo[6.1.0]non-4-yne-poly(ethylene glycol)-carboxylic acid, dibenzocyclooctyne-poly(ethylene glycol)-amine, norbornene-poly(ethylene glycol)-amine, bicyclo[6.1.0]non-4-yne-poly(ethylene glycol)-amine, dibenzocyclooctyne-poly(ethylene glycol)-hydroxyl, norbornene-poly(ethylene glycol)-hydroxyl, bicyclo[6.1.0]non-4-yne-poly(ethylene glycol)-hydroxyl, dibenzocyclooctyne-poly(ethylene glycol)-sulfonic acid, norbornene-poly(ethylene glycol)-sulfonic acid, and bicyclo[6.1.0]non-4-yne-poly(ethylene glycol)-sulfonic acid.

4. The flow cell as defined in claim 2, wherein:
the polymeric hydrogel includes terminal azide groups;
the second linker is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether;
the second end group is the zwitterionic end group; and
the zwitterionic end group is selected from the group consisting of phosphocholine, a sulfobetaine, and a carboxybetaine.

5. The flow cell as defined in claim 2, wherein:
the polymeric hydrogel includes terminal azide groups;
the second linker is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether;
the second end group is the amphoteric polymer end group; and
the amphoteric polymer end group includes both a carboxylic acid functional group and an amine functional group.

6. The flow cell as defined in claim 2, wherein:
the polymeric hydrogel includes terminal azide groups;
the second linker is poly(ethylene glycol)-dibenzocyclooctyne, poly(ethylene glycol)-norbornene, bicyclo[6.1.0]non-4-yne-poly(ethylene glycol), or bicyclo[6.1.0]non-4-yne-poly(ethylene glycol) methyl ether;
the second end group is the anionic polymer end group; and
the anionic polymer end group is poly(meth) acrylic acid.

7. The flow cell as defined in claim 1, wherein:
the passivation component is exclusively attached to the polymeric hydrogel;
the passivation component includes the dibenzocyclooctyne-functionalized passivation agent; and
the dibenzocyclooctyne-functionalized passivation agent is selected from the group consisting of dibenzocyclooctyne-acid, dibenzocyclooctyne-amine, and dibenzocyclooctyne-sulfo-amine.

8. The flow cell as defined in claim 1, wherein:
the passivation component is exclusively attached to the interstitial regions;
the first linker is poly(ethylene glycol) and further includes a silane functional group attached to the interstitial regions;
the first end group is the zwitterionic end group; and
the zwitterionic end group is selected from the group consisting of phosphocholine, a sulfobetaine, and a carboxybetaine.

9. The flow cell as defined in claim 1, wherein:
the passivation component is exclusively attached to the interstitial regions;
the first linker is poly(ethylene glycol) and further includes a silane functional group attached to the interstitial regions;
the first end group is the amphoteric polymer end group; and
the amphoteric polymer end group includes both a carboxylic acid functional group and an amine functional group.

10. The flow cell as defined in claim 1, wherein:
the passivation component is exclusively attached to the interstitial regions; and
the passivation component is selected from the group consisting of (heptadecafluoro-1,1,2,2-tetrahydrodecyl) trimethoxysilane, 3-(heptafluoroisopropoxy)propyltrimethoxysilane, nonafluorohexyltrimethoxysilane, [perfluoro(polypropyleneoxy)]methoxypropyltrimethoxysilane, 1,3-bis(trifluoropropyl)-1,1,3,3-tetramethyldisilazane, and (tridecafluoro-1,1,2,2-tetrahydrooctyl)dimethylchlorosilane.

11. The flow cell as defined in claim 1, wherein
the passivation component is exclusively attached to the interstitial regions; and
the passivation component is selected from the group consisting of N,N-bis(2-hydroxyethyl)-3-aminopropyltriethoxysilane, bis(methoxyethyl)-3-trimethoxysilylpropylammoniumchloride, carboxyethylsilanetriol disodium salt, and 3-(trihydroxysilyl)-1-propanesulfonic acid.

12. The flow cell as defined in claim 1, wherein:
the respective passivation components are attached to each of the interstitial regions and the polymeric hydrogel;
the respective passivation component attached to the interstitial regions is selected from the group consisting of 2-[methoxypoly(ethylenoxy)6-9propyl]dimethylmethoxysilane, 2-[methoxy(polyethyleneoxy)6-9propyl]trimethoxysilane, 2-[methoxy(polyethyleneoxy)9-12propyl]trimethoxysilane, 2-[methoxy(polyethyleneoxy)21-24propyl]trimethoxysilane, N,N-bis(2-hydroxyethyl)-3-aminopropyltriethoxysilane, (heptadecafluoro-1,1,2,2-tetrahydrodecyl) trimethoxysilane, 3-(heptafluoroisopropoxy)propyltrimethoxysilane, nonafluorohexyltrimethoxysilane, [perfluoro(polypropyleneoxy)]methoxypropyltrimethoxysilane, 1,3-bis(trifluoropropyl)-1,1,3,3-tetramethyldisilazane, (tridecafluoro-1,1,2,2-tetrahydrooctyl)dimethylchlorosilane, N,N-bis(2-hydroxyethyl)-3-aminopropyltriethoxysilane, bis(methoxyethyl)-3-trimethoxysilylpropylammoniumchloride, carboxyethylsilanetriol disodium salt, and 3-(trihydroxysilyl)-1-propanesulfonic acid; and
the respective passivation component attached to the polymeric hydrogel is selected from the group consisting of dibenzocyclooctyne-poly(ethylene glycol)-carboxylic acid, norbornene-poly(ethylene glycol)-carboxylic acid, bicyclo[6.1.0]non-4-yne-poly(ethylene glycol)-carboxylic acid, dibenzocyclooctyne-poly(ethylene glycol)-amine, norbornene-poly(ethylene glycol)-amine, bicyclo[6.1.0]non-4-yne-poly(ethylene glycol)-amine, dibenzocyclooctyne-poly(ethylene glycol)-hydroxyl, norbornene-poly(ethylene glycol)-hydroxyl, bicyclo[6.1.0]non-4-yne-poly(ethylene glycol)-hydroxyl, dibenzocyclooctyne-poly(ethylene glycol)-sulfonic acid, norbornene-poly(ethylene glycol)-sulfonic acid, and bicyclo[6.1.0]non-4-yne-poly(ethylene glycol)-sulfonic acid.

13. The flow cell as defined in claim 1, wherein:
the respective passivation components are attached to each of the interstitial regions and the polymeric hydrogel;
the third end group and the fourth end group are each a zwitterionic end group selected from the group consisting of phosphocholine, a sulfobetaine, and a carboxybetaine;
the third linker is poly(ethylene glycol) and further includes a silane functional group attached to the interstitial regions; and
the fourth linker is poly(ethylene glycol)-dibenzocyclooctyne, poly(ethylene glycol)-norbornene, bicyclo[6.1.0]non-4-yne-poly(ethylene glycol), bicyclo[6.1.0]non-4-yne-poly(ethylene glycol) methyl ether, or a combination of poly(ethylene glycol) and pentaerythritol.

14. The flow cell as defined in claim 1, wherein:
the respective passivation components are attached to each of the interstitial regions and the polymeric hydrogel;
the third end group and the fourth end group are each the amphoteric polymer end group;
the amphoteric polymer end group includes both a carboxylic acid functional group and an amine functional group;
the third linker is poly(ethylene glycol) and further includes a silane functional group attached to the interstitial regions; and
the fourth linker is poly(ethylene glycol)-dibenzocyclooctyne or poly(ethylene glycol)-norbornene, bicyclo[6.1.0]non-4-yne-poly(ethylene glycol), bicyclo[6.1.0]non-4-yne-poly(ethylene glycol) methyl ether, or a combination of poly(ethylene glycol) and pentaerythritol.

15.-32. (canceled)


SEQUENCE LISTING (12 sequences)

SEQ ID NO: 1 - DNA, 29 bp, synthetic construct
  source 1..29: mol_type = other DNA
  modified_base 23: mod_base = OTHER; note = Uracil
aatgatacggcgaccaccgagatctacac

SEQ ID NO: 2 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 3 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 4 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 5 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 6 - DNA, 29 bp, synthetic construct
  source 1..29: mol_type = other DNA
  modified_base 23: mod_base = OTHER; note = allyl T
aatgatacggcgaccaccgaganctacac

SEQ ID NO: 7 - DNA, 35 bp, synthetic construct
  source 1..35: mol_type = other DNA
gctggcacgtccgaacgcttcgttaatccgttgag

SEQ ID NO: 8 - DNA, 35 bp, synthetic construct
  source 1..35: mol_type = other DNA
cgtcgtctgccatggcgcttcggtggatatgaact

SEQ ID NO: 9 - DNA, 35 bp, synthetic construct
  source 1..35: mol_type = other DNA
acggccgctaatatcaacgcgtcgaatccgcaact

SEQ ID NO: 10 - DNA, 34 bp, synthetic construct
  source 1..34: mol_type = other DNA
gccgcgttacgttagccggactattcgatgcagc

SEQ ID NO: 11 - DNA, 24 bp, synthetic construct
  source 1..24: mol_type = other DNA
aggaggaggaggaggaggaggagg

SEQ ID NO: 12 - DNA, 24 bp, synthetic construct
  source 1..24: mol_type = other DNA
cctcctcctcctcctcctcctcct